SEQUENCE LISTING (10 sequences)

SEQ ID NO: 1 - protein, 11 aa, Synthetic construct
CRVVLEDIMDY

SEQ ID NO: 2 - protein, 11 aa, Synthetic construct
DGPVRRTTGDV

SEQ ID NO: 3 - protein, 11 aa, Synthetic construct
GRGSARQGVDY

SEQ ID NO: 4 - protein, 5 aa, Synthetic construct
TYGIQ

SEQ ID NO: 5 - protein, 17 aa, Synthetic construct
WIHAGTGGTKYSRKFQG

SEQ ID NO: 6 - protein, 11 aa, Synthetic construct
DKVTVWACQDN

SEQ ID NO: 7 - protein, 30 aa, Synthetic construct
EVQLVESAAEVRRPGASVKITCKASGYSFS

SEQ ID NO: 8 - protein, 14 aa, Synthetic construct
WMRQAPGQRPEWLG

SEQ ID NO: 9 - protein, 32 aa, Synthetic construct
RITITRDTSANTVYLDLNSLTSEDTAVYYCAR

SEQ ID NO: 10 - protein, 11 aa, Synthetic construct
WGQGTTVTVSS

(19) United States
(12) Patent Application Publication
(10) Pub. No.: US 2025/0085299 A1
(43) Pub. Date: Mar. 13, 2025

(54) IMMUNOASSAY DEVICE FOR DETECTING BLOOD TYPE ANTIGEN-SPECIFIC IMMUNOGLOBULIN, IMMUNOASSAY METHOD FOR DETECTING BLOOD TYPE ANTIGEN-SPECIFIC IMMUNOGLOBULIN, AND METHOD FOR COLLECTING BLOOD TYPE ANTIGEN-SPECIFIC IMMUNOGLOBULIN

(71) Applicant: UIF (University Industry Foundation), Yonsei University, Seoul (KR)

(72) Inventor: Jae Chul PYUN, Seoul (KR)

(73) Assignee: UIF (University Industry Foundation), Yonsei University, Seoul (KR)

(21) Appl. No.: 18/822,766

(22) Filed: Sep. 3, 2024

(30) Foreign Application Priority Data
Sep. 8, 2023 (KR) 10-2023-0119432

(51) Int. Cl. G01N 33/80; G01N 33/542; G01N 33/543

(52) U.S. Cl. CPC G01N 33/80; G01N 33/542; G01N 33/54386

(57) ABSTRACT

An immunoassay device for detecting blood type antigen-specific immunoglobulin, an immunoassay method for detecting blood type antigen-specific immunoglobulin, and a method for collecting blood type antigen-specific immunoglobulin are disclosed. The immunoassay device for detecting blood type antigen-specific immunoglobulin includes a substrate having a reaction space defined therein capable of receiving therein a detection target sample solution; a binding antibody positioned in the reaction space and immobilized on the substrate; a switching peptide having a peptide compound reversibly binding to a Fab region of the binding antibody and a fluorescent label binding to the peptide compound; and a fluorescence-quenching substance positioned adjacent to the fluorescent label and quenching fluorescence from the fluorescent label.

### CROSS-REFERENCE TO RELATED APPLICATION

This application claims priority to Korean Patent Application No. 10-2023-0119432 filed on Sep. 8, 2023 in the Korean Intellectual Property Office, and all the benefits accruing therefrom under 35 U.S.C. 119, the contents of which in its entirety are herein incorporated by reference.

### SEQUENCE LISTING

The instant application contains a Sequence Listing which has been submitted in XML format and is hereby incorporated by reference in its entirety. Said XML copy, created on Aug. 20, 2024, is named “NCIP.P0148US_SequenceListing. XML” and is 9,583 bytes in size.

### BACKGROUND

### Field

The present disclosure relates to an immunoassay device for detecting blood type antigen-specific immunoglobulin, an immunoassay method for detecting blood type antigen-specific immunoglobulin, and a method for collecting blood type antigen-specific immunoglobulin.

### Description of Related Art

ABO blood type classification has been widely used to classify blood to determine the possibility of blood transfusion. The ABO blood type classification is based on types of antigens of red blood cells (hereinafter “RBC”), such as A-type and B-type antigens. In the case of A-type blood, RBCs with A-type antigens and IgM specific to B-type antigens are found. In the case of B-type blood, RBCs with B-type antigens and IgM specific to A-type antigens are found. For example, when IgM specific to B-type antigens meets RBCs with B-type antigens, hemagglutination of RBCs, i.e. an immune response thereof, occurs. The same is true for A-type. Accordingly, blood transfusion between incompatible blood types is not realized in principle. In particular, ABO antigens are known to be found on the surfaces of various organs. Thus, blood type matching is required for organ transplantation.

Blood transfusions or organ transplants between people with different blood types have limitations due to the above-mentioned immune response. However, with the advancement of medical technology, in cases of blood type incompatibility, the so-called desensitization therapy may be used to significantly reduce the immune response, thereby making the blood transfusions or organ transplants between people with different blood types possible. In the desensitization therapy, plasma exchange is basically performed, and Rituximab is administered to the subject to prevent the generation of B cells that produce antibody to suppress the immune response (Non-patent Document 1). Usually, the administering of the Rituximab is first performed, and then the plasma exchange is performed.

### SUMMARY

The inventors of the present disclosure have completed a method and device for collecting immunoglobulin in plasma and sensing the remaining immunoglobulin, based on antibody and peptides for reducing the immune response that may occur during organ transplantation between different blood types.

One purpose of the present disclosure is to provide an immunoassay device for detecting blood type antigen-specific immunoglobulin that may detect immunoglobulin in one step.

Another purpose of the present disclosure is to provide an immunoassay method for detecting blood type antigen-specific immunoglobulin using the above device.

Still another purpose of the present disclosure is to provide a method for collecting blood type antigen-specific immunoglobulin present in plasma.

Purposes according to the present disclosure are not limited to the above-mentioned purpose. Other purposes and advantages according to the present disclosure that are not mentioned may be understood based on following descriptions, and may be more clearly understood based on embodiments according to the present disclosure. Further, it will be easily understood that the purposes and advantages according to the present disclosure may be realized using means shown in the claims and combinations thereof.

A first aspect of the present disclosure provides an immunoassay device for detecting blood type antigen-specific immunoglobulin, the immunoassay device comprising: a substrate having a reaction space defined therein capable of receiving therein a detection target sample solution; a binding antibody positioned in the reaction space and immobilized on the substrate; a switching peptide having a peptide compound reversibly binding to a Fab region of the binding antibody and a fluorescent label binding to the peptide compound; and a fluorescence-quenching substance positioned adjacent to the fluorescent label and quenching fluorescence from the fluorescent label, wherein the binding antibody includes one selected from the group consisting of a CDR3 including an amino acid sequence of SEQ ID NO 1, a CDR3 including an amino acid sequence of SEQ ID NO 2, and a CDR3 including an amino acid sequence of SEQ ID NO 3, and thus specifically binds to the blood type antigen-specific immunoglobulin.

In accordance with some embodiments of the first aspect, the binding antibody includes one selected from the group consisting of: a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 1; a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 2; and a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 3.

In accordance with some embodiments of the first aspect, the peptide compound has an amino acid sequence capable of specifically and reversibly binding to at least one of first to fourth framework regions of a light chain or a heavy chain of the binding antibody.

In accordance with some embodiments of the first aspect, the binding antibody includes one selected from the group consisting of: a heavy chain variable region including an amino acid sequence of SEQ ID NO 1; a heavy chain variable region including an amino acid sequence of SEQ ID NO 2; and a heavy chain variable region including an amino acid sequence of SEQ ID NO 3.

In accordance with some embodiments of the first aspect, the immunoassay device further comprises: a light source for irradiating light to the detection sample solution received in the reaction space of the substrate; and an image analysis device configured to receive fluorescence generated from the switching peptide released from the binding antibody, to generate an image of the received fluorescence, and to analyze the image to analyze an amount of the switching peptide released from the binding antibody.

In accordance with some embodiments of the first aspect, the blood type antigen-specific immunoglobulin is A-type antigen-specific IgM or B-type antigen-specific IgM.

In accordance with some embodiments of the first aspect, the fluorescent label includes at least one selected from the group consisting of rhodamine and derivatives thereof, fluorescein and derivatives thereof, coumarin and derivatives thereof, acridine and derivatives thereof, pyrene and derivatives thereof, erythrosine and derivatives thereof, eosin and derivatives thereof, 4-acetamido-4′-isothiocyanatostilbene-2,2′ disulfonic acid, fluorescein isothiocyanate (FITC), oregon green, Alex Fluoro, carboxyfluorescein (FAM), 6-carboxy-4′,5′-dichloro-2′,7′-dimethoxyfluorescein (JOE), carboxy-X-rhodamine (ROX), 6-carboxy-2′,4,4′,5′,7,7′-hexachlorofluorescein (HEX), Texas Red (sulforhodamine 101 acid chloride), 6-carboxy-2′,4,7′,7-tetrachlorofluorescein (TET), tetramethylrhodamine-isothiocyanate (TRITC), carboxytetramethylrhodamine (TAMRA), cyanine-based dyes, and siadicarbocyanine dyes.

In accordance with some embodiments of the first aspect, the fluorescence-quenching substance includes at least one selected from the group consisting of 4-(dimethylamino) azobenzene-4-carboxylic acid (DABCYL), 4-(dimethylamino) azobenzene sulfonic acid (DABSYL), blackhole quencher (BHQ), blackberry quencher (BBQ), ECLIPSE quencher, Tide quencher, a carbon nanomaterial, and a manganese dioxide nanomaterial.

A second aspect of the present disclosure provides an immunoassay device for detecting blood type antigen-specific immunoglobulin, the immunoassay device comprising: a substrate having a reaction space defined therein capable of receiving therein a detection target sample solution; a support positioned in the reaction space; a binding antibody binding to the support; a switching peptide having a peptide compound reversibly binding to a Fab region of the binding antibody and a fluorescent label binding to the peptide compound; and a fluorescence-quenching substance positioned adjacent to the fluorescent label and quenching fluorescence from the fluorescent label, wherein the binding antibody includes one selected from the group consisting of a CDR3 including an amino acid sequence of SEQ ID NO 1, a CDR3 including an amino acid sequence of SEQ ID NO 2, and a CDR3 including an amino acid sequence of SEQ ID NO 3, and thus specifically binds to the blood type antigen-specific immunoglobulin.

In accordance with some embodiments of the second aspect, the binding antibody includes one selected from the group consisting of: a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 1; a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 2; and a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 3.

In accordance with some embodiments of the second aspect, the peptide compound has an amino acid sequence capable of specifically and reversibly binding to at least one of first to fourth framework regions of a light chain or a heavy chain of the binding antibody.

In accordance with some embodiments of the second aspect, the binding antibody includes one selected from the group consisting of: a heavy chain variable region including an amino acid sequence of SEQ ID NO 1; a heavy chain variable region including an amino acid sequence of SEQ ID NO 2; and a heavy chain variable region including an amino acid sequence of SEQ ID NO 3.

In accordance with some embodiments of the second aspect, the immunoassay device further comprises: a light source for irradiating light to the detection sample solution received in the reaction space of the substrate; and an image analysis device configured to receive fluorescence generated from the switching peptide released from the binding antibody, to generate an image of the received fluorescence, and to analyze the image to analyze an amount of the switching peptide released from the binding antibody.

In accordance with some embodiments of the second aspect, the blood type antigen-specific immunoglobulin is A-type antigen-specific IgM or B-type antigen-specific IgM.

In accordance with some embodiments of the second aspect, the fluorescent label includes at least one selected from the group consisting of rhodamine and derivatives thereof, fluorescein and derivatives thereof, coumarin and derivatives thereof, acridine and derivatives thereof, pyrene and derivatives thereof, erythrosine and derivatives thereof, eosin and derivatives thereof, 4-acetamido-4′-isothiocyanatostilbene-2,2′ disulfonic acid, fluorescein isothiocyanate (FITC), oregon green, Alex Fluoro, carboxyfluorescein (FAM), 6-carboxy-4′,5′-dichloro-2′,7′-dimethoxyfluorescein (JOE), carboxy-X-rhodamine (ROX), 6-carboxy-2′,4,4′,5′,7,7′-hexachlorofluorescein (HEX), Texas Red (sulforhodamine 101 acid chloride), 6-carboxy-2′,4,7′,7-tetrachlorofluorescein (TET), tetramethylrhodamine-isothiocyanate (TRITC), carboxytetramethylrhodamine (TAMRA), cyanine-based dyes, and siadicarbocyanine dyes.

In accordance with some embodiments of the second aspect, the fluorescence-quenching substance includes at least one selected from the group consisting of 4-(dimethylamino) azobenzene-4-carboxylic acid (DABCYL), 4-(dimethylamino) azobenzene sulfonic acid (DABSYL), blackhole quencher (BHQ), blackberry quencher (BBQ), ECLIPSE quencher, Tide quencher, a carbon nanomaterial, and a manganese dioxide nanomaterial.

A third aspect of the present disclosure provides an immunoassay method for detecting blood type antigen-specific immunoglobulin, the method comprising: a first step of immobilizing a binding antibody having a switching peptide binding thereto to a substrate or immobilizing the binding antibody to the substrate, and then binding the switching peptide to the binding antibody, and binding a quenching substance to the binding antibody or the substrate; a second step of treating the binding antibody with a detection target sample solution; and a third step of irradiating light to the detection target sample solution after the treatment, and quantitatively analyzing a target antigen in the detection target sample solution based on fluorescence generated from a fluorescent label of the switching peptide released from the binding antibody, wherein the binding antibody includes one selected from the group consisting of a CDR3 including an amino acid sequence of SEQ ID NO 1, a CDR3 including an amino acid sequence of SEQ ID NO 2, and a CDR3 including an amino acid sequence of SEQ ID NO 3, and thus specifically binds to the blood type antigen-specific immunoglobulin, wherein the switching peptide includes a peptide compound having an amino acid sequence of capable selectively and reversibly binding to a Fab (Fragment antigen-binding) region of the binding antibody, and the fluorescent label binding thereto and emitting fluorescent light.

In accordance with some embodiments of the third aspect, in the second step, when the blood type antigen-specific immunoglobulin is present in the detection target sample solution, and thus the blood type antigen-specific immunoglobulin binds to the binding antibody, the switching peptide is quantitatively released from the binding antibody depending on an amount of the blood type antigen-specific immunoglobulin having reacted with the binding antibody.

A fourth aspect of the present disclosure provides an immunoassay method for detecting blood type antigen-specific immunoglobulin, the immunoassay method comprising: a first step of immobilizing a binding antibody having a switching peptide binding thereto to a support or immobilizing the binding antibody to the support and then binding the switching peptide to the binding antibody, and then, binding a quenching substance to the binding antibody or the support; a second step of injecting the support having the binding antibody binding thereto and a detection target sample solution into a reaction space of a substrate; and a third step of irradiating light to the detection target sample solution and quantitatively analyzing a target antigen in the detection target sample solution, based on fluorescence generated from a fluorescent label of the switching peptide released from the binding antibody, wherein the binding antibody includes one selected from the group consisting of a CDR3 including an amino acid sequence of SEQ ID NO 1, a CDR3 including an amino acid sequence of SEQ ID NO 2, and a CDR3 including an amino acid sequence of SEQ ID NO 3, and thus specifically binds to the blood type antigen-specific immunoglobulin, wherein the switching peptide includes a peptide compound having an amino acid sequence of capable selectively and reversibly binding to a Fab (Fragment antigen-binding) region of the binding antibody, and the fluorescent label binding thereto and emitting fluorescent light.

In accordance with some embodiments of the fourth aspect, in the second step, when the blood type antigen-specific immunoglobulin is present in the detection target sample solution, and thus the blood type antigen-specific immunoglobulin binds to the binding antibody, the switching peptide is quantitatively released from the binding antibody depending on an amount of the blood type antigen-specific immunoglobulin having reacted with the binding antibody.

A fifth aspect of the present disclosure provides a method for collecting blood type antigen-specific immunoglobulin, the method comprising: adding a bead having an antibody or a peptide immobilized on a surface thereof specifically binding to the blood type antigen-specific immunoglobulin into a sample containing the blood type antigen-specific immunoglobulin to induce a reaction between the blood type antigen-specific immunoglobulin and the antibody or the peptide; and collecting the bead from the sample, wherein the binding antibody includes one selected from the group consisting of a CDR3 including an amino acid sequence of SEQ ID NO 1, a CDR3 including an amino acid sequence of SEQ ID NO 2, and a CDR3 including an amino acid sequence of SEQ ID NO 3, and thus specifically binds to the blood type antigen-specific immunoglobulin.

In accordance with some embodiments of the fifth aspect, the bead includes nickel (Ni), copper (Cu), zinc (Zn), or cobalt (Co)-containing magnetic particles having a chelating group including nitrilotriacetic acid (NTA) or iminodiacetic acid (IDA) modified on surfaces thereof.

In accordance with some embodiments of the fifth aspect, the bead is made of a magnetic material, wherein the bead is collected from the sample using a magnetic field.

The immunoassay method for detecting the blood type antigen-specific immunoglobulin according to an embodiment of the present disclosure may detect immunoglobulin in plasma in one step.

The immunoassay method for detecting the blood type antigen-specific immunoglobulin may be implemented using the immunoassay device for detecting the blood type antigen-specific immunoglobulin according to an embodiment of the present disclosure.

The immunoglobulin may be collected from plasma using the method for collecting the blood type antigen-specific immunoglobulin according to an embodiment of the present disclosure.

In addition to the effects as described above, specific effects in accordance with the present disclosure will be described together with following detailed descriptions for carrying out the disclosure.

### BRIEF DESCRIPTION OF DRAWINGS

FIG. 1 is a drawing schematically illustrating an immunoassay device for detecting blood type antigen-specific immunoglobulin according to an embodiment of the present disclosure.

FIG. 2 is a drawing schematically illustrating another aspect of the immunoassay device for detecting blood type antigen-specific immunoglobulin according to an embodiment of the present disclosure.

FIG. 3 is a flow chart schematically illustrating an immunoassay method for detecting blood type antigen-specific immunoglobulin according to an embodiment of the present disclosure.

FIG. 4 is a flow chart schematically illustrating an immunoassay method for detecting blood type antigen-specific immunoglobulin according to another embodiment of the present disclosure.

FIG. 5 is a flow chart schematically illustrating a method for collecting blood type antigen-specific immunoglobulin according to an embodiment of the present disclosure.

FIGS. 6A to 22 are diagrams illustrating experimental procedures and/or experimental results according to experimental examples of the present disclosure.

### DETAILED DESCRIPTIONS

Advantages and features of the present disclosure, and a method of achieving the advantages and features will become apparent with reference to embodiments described later in detail together with the accompanying drawings. However, the present disclosure is not limited to the embodiments as disclosed under, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

For simplicity and clarity of illustration, elements in the drawings are not necessarily drawn to scale. The same reference numbers in different drawings represent the same or similar elements, and as such perform similar functionality. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included in the spirit and scope of the present disclosure as defined by the appended claims.

A shape, a size, a ratio, an angle, a number, etc. disclosed in the drawings for describing embodiments of the present disclosure are illustrative, and the present disclosure is not limited thereto. The same reference numerals refer to the same elements herein. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in this specification, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

In addition, it will also be understood that when a first element or layer is referred to as being present “on” a second element or layer, the first element may be disposed directly on the second element or may be disposed indirectly on the second element with a third element or layer being disposed between the first and second elements or layers. It will be understood that when an element or layer is referred to as being “connected to”, or “connected to” another element or layer, it may be directly on, connected to, or connected to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

Further, as used herein, when a layer, film, region, plate, or the like is disposed “on” or “on a top” of another layer, film, region, plate, or the like, the former may directly contact the latter or still another layer, film, region, plate, or the like may be disposed between the former and the latter. As used herein, when a layer, film, region, plate, or the like is directly disposed “on” or “on a top” of another layer, film, region, plate, or the like, the former directly contacts the latter and still another layer, film, region, plate, or the like is not disposed between the former and the latter. Further, as used herein, when a layer, film, region, plate, or the like is disposed “below” or “under” another layer, film, region, plate, or the like, the former may directly contact the latter or still another layer, film, region, plate, or the like may be disposed between the former and the latter. As used herein, when a layer, film, region, plate, or the like is directly disposed “below” or “under” another layer, film, region, plate, or the like, the former directly contacts the latter and still another layer, film, region, plate, or the like is not disposed between the former and the latter.

In descriptions of temporal relationships, for example, temporal precedent relationships between two events such as “after”, “subsequent to”, “before”, etc., another event may occur therebetween unless “directly after”, “directly subsequent” or “directly before” is not indicated.

When a certain embodiment may be implemented differently, a function or an operation specified in a specific block may occur in a different order from an order specified in a flowchart. For example, two blocks in succession may be actually performed substantially concurrently, or the two blocks may be performed in a reverse order depending on a function or operation involved.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described under could be termed a second element, component, region, layer or section, without departing from the spirit and scope of the present disclosure.

The features of the various embodiments of the present disclosure may be partially or entirely combined with each other, and may be technically associated with each other or operate with each other. The embodiments may be implemented independently of each other and may be implemented together in an association relationship.

In interpreting a numerical value, the value is interpreted as including an error range unless there is no separate explicit description thereof.

It will be understood that when an element or layer is referred to as being “connected to”, or “connected to” another element or layer, it may be directly on, connected to, or connected to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

The features of the various embodiments of the present disclosure may be partially or entirely combined with each other, and may be technically associated with each other or operate with each other. The embodiments may be implemented independently of each other and may be implemented together in an association relationship.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “embodiments,” “examples,” “aspects, and the like should not be construed such that any aspect or design as described is superior to or advantageous over other aspects or designs.

Further, the term ‘or’ means ‘inclusive or’ rather than ‘exclusive or’. That is, unless otherwise stated or clear from the context, the expression that ‘x uses a or b’ means any one of natural inclusive permutations.

The terms used in the description below have been selected as being general and universal in the related technical field. However, there may be other terms than the terms depending on the development and/or change of technology, convention, preference of technicians, etc. Therefore, the terms used in the description below should not be understood as limiting technical ideas, but should be understood as examples of the terms for describing embodiments.

Further, in a specific case, a term may be arbitrarily selected by the applicant, and in this case, the detailed meaning thereof will be described in a corresponding description section. Therefore, the terms used in the description below should be understood based on not simply the name of the terms, but the meaning of the terms and the contents throughout the Detailed Descriptions.

As used herein, the term “antibody” encompasses all forms of antibody known to date in the most comprehensive definition. That is, the antibody encompasses various antibody structures, including but not limited to a monoclonal antibody, a polyclonal antibody, a multi-specific antibody (e.g., a bispecific antibody), a full-length antibody, and an antigen-binding fragment thereof, as long as they exhibit antigen-binding activity. The term “antibody” includes a conventional 4-chain antibody, a single domain antibody, and an antigen-binding fragment thereof.

The single domain antibody (sdAb) according to the present disclosure may include, without limitation, engineered domains and single domain scaffolds in addition to heavy chain variable domains from heavy chain-only antibodies (for example, VHH in Camelidae (variable domain of the heavy chain of a heavy chain antibody)); light chains derived from conventional four-chain antibodies; binding molecules naturally devoid of a single domain (for example, VH or VL); humanized heavy chain only antibodies; human single domain antibodies produced by transgenic mice or rats expressing human heavy chain segments; and those derived from antibodies. The sdAb may be derived from any species, including but not limited to mouse, rat, human, camel, llama, lamprey, fish, shark, goat, rabbit, and bovine. The sdAb may also include naturally occurring sdAb molecules from species other than Camelidae.

Furthermore, the sdAb is derived from naturally occurring single domain antigen binding molecules known as heavy chain antibodies devoid of light chains. Such single domain molecules are disclosed, for example, in WO 94/04678 and Hamers-Casterman, et al., (1993) Nature 363:446-448. Variable domains derived from heavy chain molecules naturally devoid of light chains are known herein as VHHs to distinguish these from the conventional VHs of four chain immunoglobulins. Such VHH molecules may be derived from antibodies produced in species of Camelidae such as camels, llamas, vicunas, dromedary camels, alpacas and guanacos. Species other than Camelidae can produce heavy chain molecules naturally devoid of light chains, and such VHHs are within the scope of the present disclosure.

In addition, the sdAb according to the present disclosure may be a chimeric antibody. Certain chimeric antibodies are described in, for example, U.S. Pat. No. 4,816,567; and Morrison et al., Proc. Natl. Acad. Sci. USA, 81:6851-6855 (1984)). In some embodiments, a chimeric antibody may comprise a non-human variable region (for example, a variable region derived from a species of Camelidae, such as a llama) and a human constant region. In addition, a chimeric antibody may be humanized. Typically, a non-human antibody is humanized to reduce immunogenicity to humans but maintain the specificity and affinity of the parental non-human antibody. Generally, a humanized antibody comprises one or more variable domains in which CDRs (or portions thereof) are derived from non-human antibodies and the FRs (or portions thereof) are derived from human antibody sequences. A humanized antibody optionally also will comprise at least a portion of a human constant region. In some embodiments, some FR residues in a humanized antibody are substituted with the corresponding residues from a non-human antibody (for example, an antibody from which the CDR residues are derived), for example, in order to restore or improve antibody specificity or affinity.

As used herein, the term “Fv (Variable region fragment)” refers to the antigen binding region of immunoglobulin G, and refer to a minimal antibody fragment composed of three CDR regions (CDR1, CDR2 and CDR3) and a framework region (FR) between the CDR regions, and having only a heavy chain variable region and/or a light chain variable region. Recombinant techniques for producing Fv are described in international patent application publication No. WO88/10649, etc.

As used herein, the term “CDR (complementarity determining region)” refers to an amino acid residue of an antibody variable domain that is necessary for antigen binding. Each variable domain typically has three CDR regions, identified as CDR1, CDR2, and CDR3. Furthermore, the CDR of the heavy chain variable region may be referred to as HCDR.

As used herein, the term “surface expression (auto display)” is a technique for expressing (displaying) a variant polypeptide as a fusion protein with at least a portion of an envelope protein on a surface of an organism such as phage or E. coli. The utility of the surface expression lies in the fact that the technique can quickly and efficiently sort out sequences that bind to a target antigen with high affinity on a large library of randomized protein variants. Expressing peptide and protein libraries on organisms has been used to screen millions of polypeptides for polypeptides with specific binding properties. In particular, it is important to generate a diverse library of antibody or antigen-binding proteins in high-affinity antibody isolation. The CDR3 region has been found to often participate in antigen binding. The CDR3 region on the heavy chain is considerably diverse in terms of size, sequence, and structural stereoscopic form. Thus, various libraries may be prepared using the CDR3 region.

“Nucleic acid” has a meaning that comprehensively includes DNA (gDNA and cDNA) and RNA molecules. The nucleotides which are the basic structural units of nucleic acids include not only natural nucleotides but also analogues of the natural nucleotides in which sugar or base sites thereof are modified. The sequence of the nucleic acid encoding the heavy chain and light chain variable regions of the present disclosure may be modified. The modifications include addition, deletion, or non-conservative or conservative substitution of nucleotides.

The amino acid sequence for the antibody of the present disclosure or the antigen binding fragment thereof or the nucleic acid encoding the amino acid sequence is interpreted to also include a sequence that exhibits substantial identity with the sequence described in SEQ ID NO. The substantial identity means a sequence that exhibits 90% or greater homology, most preferably 95% or greater homology, 96% or greater, 97% or greater, 98% or greater, or 99% or greater homology when the sequence of the present disclosure and any other sequence are aligned with each other to the greatest possible extent and the alignment is analyzed using an algorithm commonly used in the art.

Based on the above definition, the antibody or antigen-binding fragment thereof according to the present disclosure may have a homology of 90%, 91%, 92%, 93%, 94%, 95%, 96%, 97%, 98%, or 99% or greater compared to the sequence disclosed herein or the entirety thereof. Such homology can be determined through sequence comparison and/or alignment by methods known in the art. For example, the percentage sequence homology of the nucleic acid or protein according to the present disclosure may be determined using a sequence comparison algorithm (i.e., BLAST or BLAST 2.0), manual alignment, or visual inspection.

DNA encoding the antibody may be readily isolated or synthesized using conventional procedures (e.g., by using an oligonucleotide probe that specifically binds to DNA encoding the heavy and light chains of the antibody). Many vectors are available. A vector component typically includes, but is not limited to, one or more of a signal sequence, an origin of replication, one or more marker genes, an enhancer element, a promoter, and a transcription termination sequence.

As used herein, the term “vector” refers to a means for expressing target genes in host cells, and includes plasmid vectors, cosmid vectors, and viral vectors such as bacteriophage vectors, adenovirus vectors, retroviral vectors and adeno-associated viral vectors. In the vector, the nucleic acid encoding the antibody is operably linked to promoters, transcription termination sequences or the like. The term “operably linked” means a functional linkage between a nucleic acid expression regulation sequence (e.g., an array of promoter, signal sequence or transcription regulator binding sites) and another nucleic acid sequence, and enables the regulation sequence to regulate the transcription and/or translation of the other nucleic acid sequence.

When the vector uses the prokaryotic cell as the host, the vector generally includes a strong promoter capable of driving transcription (e.g., tac promoter, lac promoter, lacUV5 promoter, lpp promoter, rac5 promoter, amp promoter, recA promoter, SP6 promoter, trp promoter, and T7 promoter, etc.), a ribosome binding site for translation initiation, and a transcription/translation termination sequence. Furthermore, for example, when the vector uses a eukaryotic cell as the host, the vector includes promoters derived from the genome of mammalian cells (e.g., metallothionein promoter, actin promoter, human hemoglobin promoter and human muscle creatine promoter) or promoters derived from mammalian viruses (e.g., adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, promoter of Moloney virus, promoter of Epstein-Barr virus (EBV) and promoter of Rous sarcoma virus (RSV)), and generally has a polyadenylation sequence as a transcription termination sequence.

Optionally, the vector may be fused with another sequence in order to facilitate purification of the antibody expressed therefrom. The sequence to be fused therewith includes, for example, glutathione S-transferase (Pharmacia, USA), maltose-binding protein (NEB, USA), FLAG (IBI, USA), 6×His (hexahistidine; Qiagen, USA) and the like.

The vector includes antibiotic-resistance genes commonly used in the art as selectable markers, and examples thereof include genes conferring resistance to ampicillin, gentamycin, carbenicillin, chloramphenicol, streptomycin, kanamycin, geneticin, neomycin and tetracycline.

Still another aspect of the present disclosure is directed to a cell transfected with the above-mentioned vector. The cell used to produce the antibody of the present disclosure may be a prokaryote, yeast or higher eukaryotic cell, but is not limited thereto. Prokaryotic host cells such as Escherichia coli, the genus Bacillus such as Bacillus subtilis and Bacillus thuringiensis, Streptomyces, Pseudomonas, Proteus mirabilis, and Staphylococcus may be used.

The cells may be cultured in various media. Any commercially available media may be used as a culture medium. Any other necessary supplements known to the skilled person may be contained the medium in appropriate concentrations. Culture conditions, such as temperature, pH, etc., are already used with the host cell selected for expression and will be apparent to the skilled person.

As used herein, the term “transformation” means introducing a vector containing a nucleic acid encoding a target protein into a host cell so that the protein encoded by said nucleic acid may be expressed in the host cell. The transformed nucleic acid includes any one, whether it is inserted within the chromosome of the host cell or located outside the chromosome, as long as it may be expressed in the host cell. Furthermore, the nucleic acid includes DNA and RNA encoding the target protein. The nucleic acid may be introduced in any form as long as it may be introduced into the host cell and expressed. For example, the nucleic acid may be introduced into the host cell in the form of an expression cassette as a genetic construct containing all the elements necessary for its own expression. The above expression cassette typically includes a promoter, a transcription termination signal, a ribosome binding site, and a translation termination signal that are operably linked to the above nucleic acid. The above expression cassette may be in the form of an expression vector capable of self-replication. Furthermore, the above nucleic acid may be introduced into a host cell in its own form and operably linked to a sequence required for expression in the host cell.

The recovery of the antibody or the antigen binding fragment thereof may be accomplished by removing impurities, for example, via centrifugation or ultrafiltration, and purifying the resultant product, for example, using affinity chromatography. Additional other purification techniques, for example, anion or cation exchange chromatography, hydrophobic interaction chromatography, hydroxylapatite chromatography, etc. may be used.

The sequences according to SEQ ID NO used throughout the present disclosure are as follows.

TABLE 1

| | Sequence | |
|---|---|---|
| SEQ ID NO | name | Sequence |
| 1 | A28 | CRVVLEDIMDY |
| 2 | B6 | DGPVRRTTGDV |
| 3 | B22 | GRGSARQGVDY |
| 4 | CDR1 | TYGIQ |
| 5 | CDR2 | WIHAGTGGTKYSRKFQG |
| 6 | template | DKVTVWACQDN |
| 7 | FR1 | EVOLVESAAEVRRPGASVKITCKASGYSFS |
| 8 | FR2 | WMRQAPGQRPEWLG |
| 9 | FR3 | RITITRDTSANTVYLDLNSLTSEDTAVYYCAR |
| 10 | FR4 | WGQGTTVTVSS |

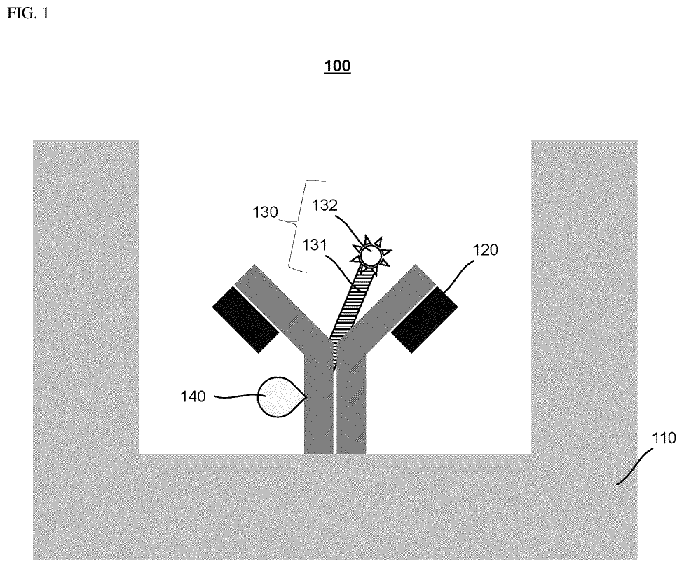

FIG. 1 is a schematic diagram of an immunoassay device for detecting blood type antigen-specific immunoglobulin according to an embodiment of the present disclosure.

Referring to FIG. 1, an immunoassay device 100 for detecting blood type antigen-specific immunoglobulin according to an embodiment of the present disclosure comprises: a substrate 110 having a reaction space defined therein capable of receiving therein a detection target sample solution; a binding antibody 120 positioned in the reaction space and immobilized on the substrate 110; a switching peptide 130 having a peptide compound 131 reversibly binding to a Fab region of the binding antibody 120 and a fluorescent label 132 binding to the peptide compound 131; and a fluorescence-quenching substance 140 that is arranged adjacent to the fluorescent label 132 and quenches fluorescence from the fluorescent label 132, wherein the binding antibody 120 may include one selected from a group consisting of a CDR3 including an amino acid sequence of SEQ ID NO 1, a CDR3 including an amino acid sequence of SEQ ID NO 2, and a CDR3 including an amino acid sequence of SEQ ID NO 3.

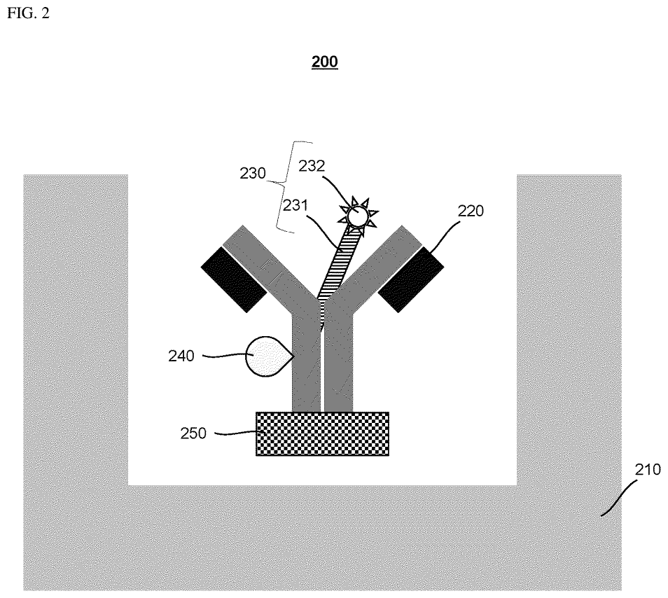

FIG. 2 is a diagram schematically illustrating another aspect of an immunoassay device for detecting blood type antigen-specific immunoglobulin according to an embodiment of the present disclosure.

Referring to FIG. 2, an immunoassay device 200 for detecting blood type antigen-specific immunoglobulin according to an embodiment of the present disclosure comprises: a substrate 210 having a reaction space defined therein capable of receiving therein a detection target sample solution; a support 250 positioned in the reaction space; a binding antibody 220 binding to the support 250; a switching peptide 230 including a peptide compound 231 reversibly binding to a Fab region of the binding antibody 220 and a fluorescent label 232 binding to the peptide compound 231; and a fluorescence-quenching substance 240 that is positioned adjacent to the fluorescent label 232 and quenches fluorescence from the fluorescent label 232, wherein the binding antibody 220 may include one selected from a group consisting of a CDR3 including an amino acid sequence of SEQ ID NO 1, a CDR3 including an amino acid sequence of SEQ ID NO 2, and a CDR3 including an amino acid sequence of SEQ ID NO 3.

Referring to FIG. 1 and FIG. 2, in one embodiment, the binding antibody 120 or 220 may include any one selected from the group consisting of: a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 1; a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 2; and a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 3;

In one embodiment, the peptide compound 131 or 231 may have an amino acid sequence that may specifically and reversibly bind to one or more of first to fourth framework regions of the light chain or the heavy chain of the binding antibody 120 or 220.

In one embodiment, the binding antibody 120 or 220 may include one selected from the group consisting of a heavy chain variable region including an amino acid sequence of SEQ ID NO 1; a heavy chain variable region including an amino acid sequence of SEQ ID NO 2; and a heavy chain variable region including an amino acid sequence of SEQ ID NO 3.

In one embodiment, the immunoassay device 100 or 200 for detecting blood type antigen-specific immunoglobulin according to one embodiment of the present disclosure may further include a light source that irradiates light to the analysis sample solution received in the reaction space of the substrate; and an image analysis device that receives fluorescence generated from the switching peptide released from the binding antibody 120 or 220 and generates an image thereof, and analyzes the image to analyze an amount of the switching peptide released from the binding antibody 120 or 220.

In one embodiment, the blood type antigen-specific immunoglobulin may be A-type antigen-specific IgM or B-type antigen-specific IgM.

In one embodiment, the fluorescent label 132 or 232 may include at least one selected from the group consisting of rhodamine and derivatives thereof, fluorescein and derivatives thereof, coumarin and derivatives thereof, acridine and derivatives thereof, pyrene and derivatives thereof, erythrosine and derivatives thereof, eosin and derivatives thereof, 4-acetamido-4′-isothiocyanatostilbene-2,2′ disulfonic acid, fluorescein isothiocyanate (FITC), oregon green, Alex Fluoro, carboxyfluorescein (FAM), 6-carboxy-4′,5′-dichloro-2′,7′-dimethoxyfluorescein (JOE), carboxy-X-rhodamine (ROX), 6-carboxy-2′,4,4′,5′,7,7′-hexachlorofluorescein (HEX), Texas Red (sulforhodamine 101 acid chloride), 6-carboxy-2′,4,7′,7-tetrachlorofluorescein (TET), tetramethylrhodamine-isothiocyanate (TRITC), carboxytetramethylrhodamine (TAMRA), cyanine-based dyes, and siadicarbocyanine dyes.

In one embodiment, the fluorescence-quenching substance 140 or 240 may include at least one selected from the group consisting of 4-(dimethylamino) azobenzene-4-carboxylic acid (DABCYL), 4-(dimethylamino) azobenzene sulfonic acid (DABSYL), blackhole quencher (BHQ), blackberry quencher (BBQ), ECLIPSE quencher, Tide quencher, a carbon nanomaterial, and a manganese dioxide nanomaterial.

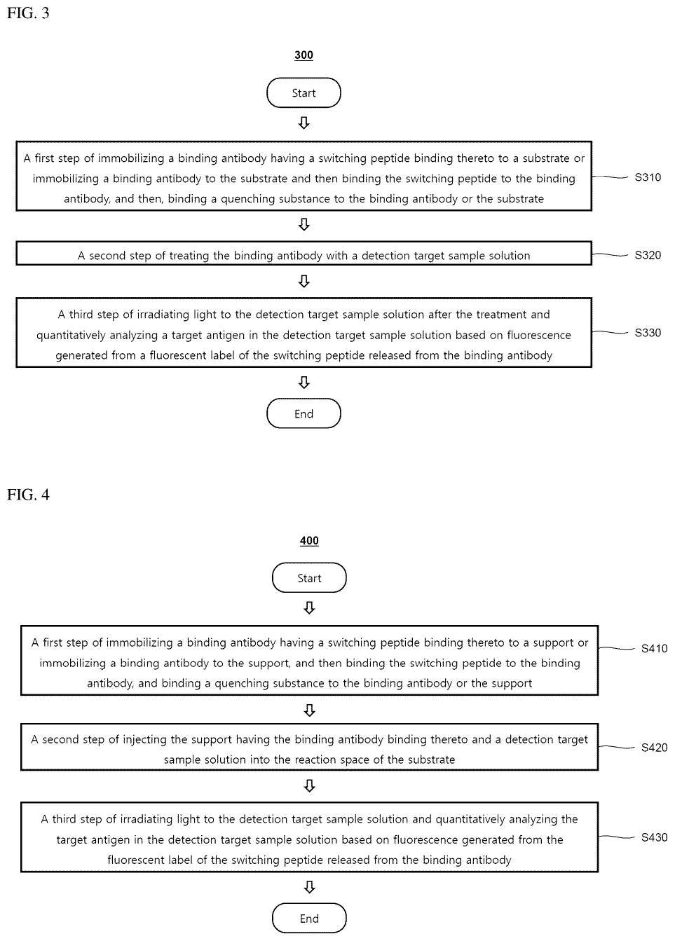

FIG. 3 is a flow chart for schematically illustrating an immunoassay method for detecting blood type antigen-specific immunoglobulin according to an embodiment of the present disclosure.

Referring to FIG. 3, an immunoassay method 300 for detecting blood type antigen-specific immunoglobulin according to an embodiment of the present disclosure may include a first step of immobilizing a binding antibody having a switching peptide binding thereto to a substrate or immobilizing a binding antibody to the substrate and then binding the switching peptide to the binding antibody, and then, binding a quenching substance to the binding antibody or the substrate in S310; a second step of treating the binding antibody with a detection target sample solution in S320; and a third step of irradiating light to the detection target sample solution after the treatment and quantitatively analyzing a target antigen in the detection target sample solution based on fluorescence generated from a fluorescent label of the switching peptide released from the binding antibody.

In one embodiment, the binding antibody may include one selected from the group consisting of the CDR3 including an amino acid sequence of SEQ ID NO 1, the CDR3 including an amino acid sequence of SEQ ID NO 2, and the CDR3 including an amino acid sequence of SEQ ID NO 3 and thus specifically binds to blood type antigen-specific immunoglobulin. The switching peptide may include a peptide compound having an amino acid sequence that may selectively and reversibly bind to the Fab (Fragment antigen-binding) region of the binding antibody, and a fluorescent label that binds thereto and emit fluorescent light.

FIG. 4 is a flow chart for schematically illustrating an immunoassay method for detecting blood type antigen-specific immunoglobulin according to another embodiment of the present disclosure.

Referring to FIG. 4, an immunoassay method 400 for detecting blood type antigen-specific immunoglobulin according to an embodiment of the present disclosure may include: a first step of immobilizing a binding antibody having a switching peptide binding thereto to a support or immobilizing a binding antibody to the support, and then binding the switching peptide to the binding antibody, and binding a quenching substance to the binding antibody or the support in S410; a second step of injecting the support having the binding antibody binding thereto and a detection target sample solution into the reaction space of the substrate in S420; and a third step of irradiating light to the detection target sample solution and quantitatively analyzing the target antigen in the detection target sample solution based on fluorescence generated from the fluorescent label of the switching peptide released from the binding antibody in S430.

In one embodiment, the binding antibody may include one selected from the group consisting of the CDR3 including an amino acid sequence of SEQ ID NO 1, the CDR3 including an amino acid sequence of SEQ ID NO 2, and the CDR3 including an amino acid sequence of SEQ ID NO 3 and thus specifically binds to blood type antigen-specific immunoglobulin. The switching peptide may include a peptide compound having an amino acid sequence that may selectively and reversibly bind to the Fab (Fragment antigen-binding) region of the binding antibody, and a fluorescent label that binds thereto and emit fluorescent light.

Referring to FIG. 3 and FIG. 4, in one embodiment, in the second step S320 or S420, when the blood type antigen-specific immunoglobulin is present in the detection target sample solution, and thus the blood type antigen-specific immunoglobulin binds to the binding antibody, the switching peptide may be quantitatively released from the binding antibody depending on the amount of the blood type antigen-specific immunoglobulin that has reacted with the binding antibody.

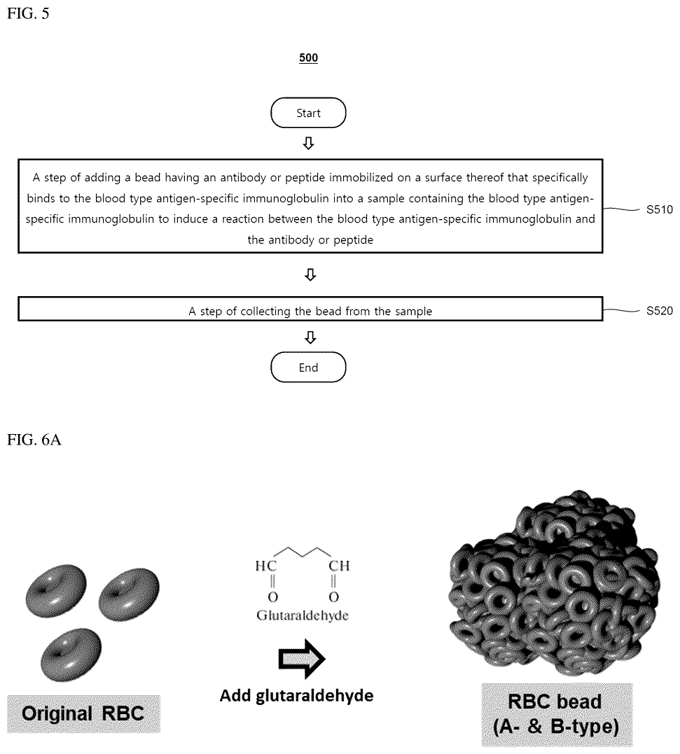

FIG. 5 is a flow chart for schematically illustrating a method for collecting blood type antigen-specific immunoglobulin according to an embodiment of the present disclosure.

Referring to FIG. 5, a method 500 for collecting blood type antigen-specific immunoglobulin according to an embodiment of the present disclosure may include a step of adding a bead having an antibody or peptide immobilized on a surface thereof that specifically binds to the blood type antigen-specific immunoglobulin into a sample containing the blood type antigen-specific immunoglobulin to induce a reaction between the blood type antigen-specific immunoglobulin and the antibody or peptide in S510; and a step of collecting the bead from the sample in S520.

In one embodiment, the binding antibody may include any one selected from the group consisting of a CDR3 including an amino acid sequence of SEQ ID NO 1, a CDR3 including an amino acid sequence of SEQ ID NO 2, and a CDR3 including an amino acid sequence of SEQ ID NO 3 and thus may specifically bind to the blood type antigen-specific immunoglobulin.

In one embodiment, the bead may include nickel (Ni), copper (Cu), zinc (Zn), or cobalt (Co)-containing magnetic particles having a chelating group such as nitrilotriacetic acid (NTA) or iminodiacetic acid (IDA) modified on surfaces thereof.

In one embodiment, the bead may be made of a magnetic material, and the beads may be collected from the sample using a magnetic field.

The following describes examples of the present disclosure. However, the examples as described below are only some embodiments of the present disclosure, and the scope of the present disclosure is not limited to the examples as set forth below.

Screening of Mimotopes of A-Type and B-Type Antigens from Fv Library

IgM, which has binding affinity to each of the A-type and B-type antigens of RBCs, is well known to induce RBC hemagglutination when plasma with ABO blood types mismatching therewith is mixed therewith. In the present disclosure, the Fv-antibody with binding affinity to IgM specific to A-type antigen and B-type antigen were selected from the Fv library. Since the selected Fv exhibited binding to IgM specific to A-type antigen (and B-type antigen), it was expected that the selected Fv had a function corresponding to the mimotope of A-type and B-type antigen in RBC, and the hemagglutination of RBCs could be effectively prevented using the selected Fv (mimotope).

The Fv library was prepared as described above. The Fv-antibody was composed of the VH of immunoglobulin G (IgG) composed of three CDR3 regions and frame regions (FRs). For the production of the Fv library, the amino acid sequence of the CDR3 region (11 residues) was randomized by site-directed mutagenesis. The diversity of the Fv library was estimated to be 105 or greater clones/libraries. The Fv library was expressed on the outer membrane of E. coli using surface expression (auto display) technology. The high expression yield was estimated to be 105 or greater Fv/E. coli, and 90% or greater of E. coli along with the total number of E. coli were estimated to surface-express Fv antibody.

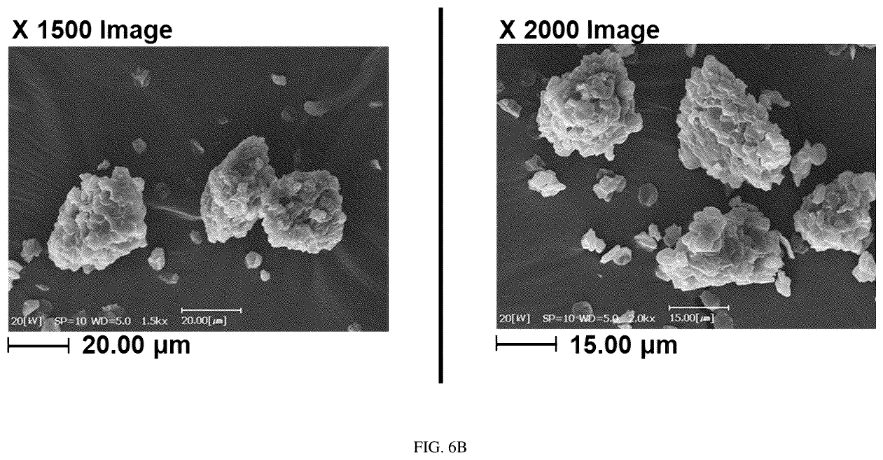

For screening of the Fv library, purified IgM specific to the A-type antigen and B-type antigen was prepared from AB-type human plasma using RBC beads. As shown in FIG. 6, RBC beads were prepared by the steps of 1) glutaraldehyde treatment, 2) lyophilization, and (3) grinding into microbeads with a diameter of approximately 50 to 100 μm. Isolation of IgM specific to A-type antigen (and B-type antigen) was performed from AB-type human plasma and crude IgM extract from Sihdia Co (Singyang Diagnostics, Korea) using RBC beads.

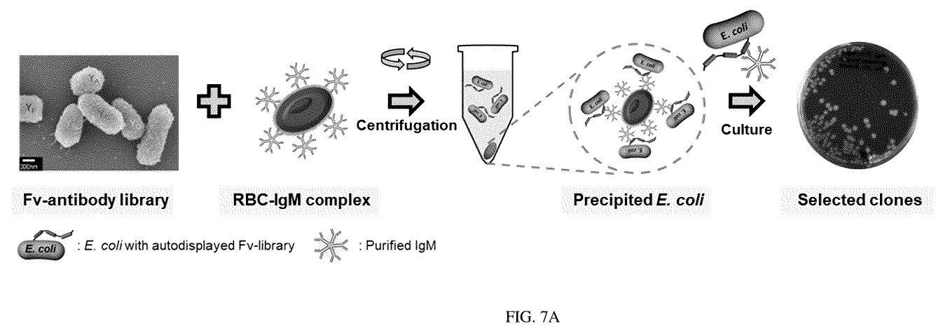

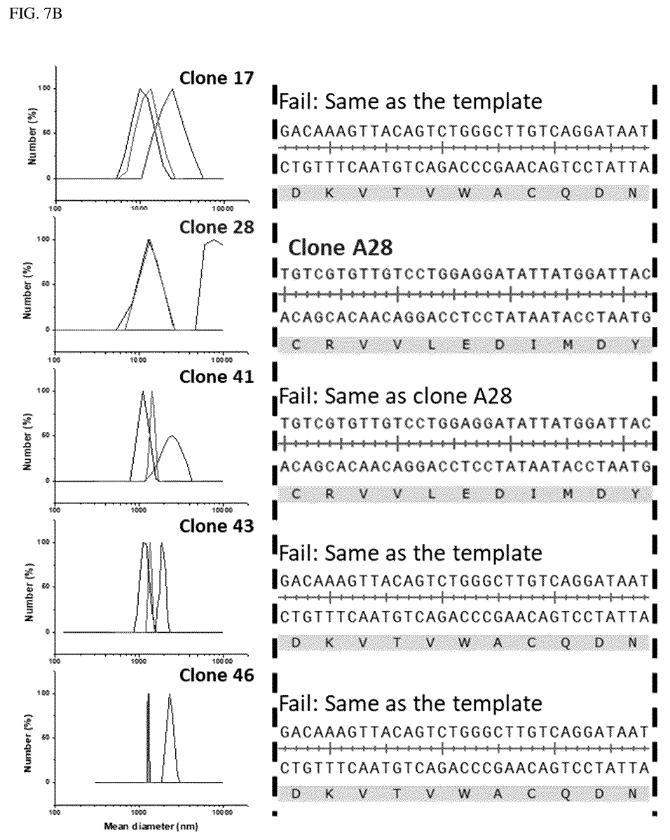

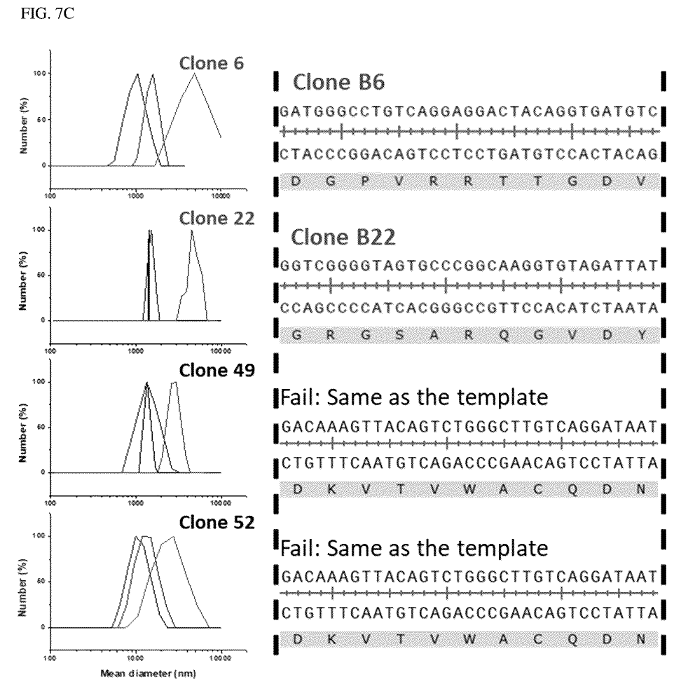

The screening of the mimotope of the A-type and B-type antigens in RBC was performed using the Fv library including purified RBC binding to IgM. Since IgM is an octamer structure of IgGs, it may have an empty binding pocket after binding to RBC. In the first step, RBC binding to IgM was prepared by mixing purified IgM specific to A-type antigen (and B-type antigen) and RBC of the A-type antigen with each other in an excess amount. As shown in FIG. 7A, RBC binding to IgM was mixed with the Fv library. In order to remove RBCs and E. coli cells of the Fv library which did not bind to each other, RBCs and E. coli cells of the Fv library were separated from each other via centrifugation. The separated RBC was cultured on an agar plate, and bound E. coli could form clusters on the agar plate, which had binding affinity to the IgM binding to RBC. Several clones were selected from the agar plate, and the binding affinity of a specific type of antigen to RBC was analyzed using dynamic laser scattering (DLS). In general, DLS may measure the scattering of monochromatic light to measure the hydrodynamic radius of a particle. To measure the affinity between E. coli and RBC, E. coli cultured in different colonies (clones) reacted with A-type RBC (blue), B-type RBC (red), and PBS (black). When E. coli binds to RBC with an average diameter of 10 μm, an increase in the size of the RBC may occur, and the change may be observed by DLS.

In the case of A-type, as shown in FIG. 7B, five clones reacted with A-type RBC (blue) and a change in size thereof was observed. In the case of B-type RBC (red) and PBS (black), no significant change occurred in the DLS signal. After genetic analysis of the CDR3 region of the Fv antibody, only one clone (clone number 28) was determined to have a valid mutation sequence, and the other four clones were determined to have the same genetic sequence as the template sequence.

In the case of B-type, as shown in FIG. 7C, four clones reacted with B-type RBC (red) and size change thereof was observed. In the case of A-type RBC (blue) and PBS (black), no significant changes occurred in the DLS signal. After genetic analysis of the CDR3 region of the Fv antibody, two clones (clone numbers 6 and 22) were determined to have a valid mutation sequence, and the other two clones were determined to have the same genetic sequence as the template sequence. The amino acid sequences of the CDR3 region of the selected clones are shown in Table 2.

TABLE 2

| SEQ ID NO | Sequence Name | Sequence |
|---|---|---|
| 1 | A28 | CRVVLEDIMDY |
| 2 | B6 | DGPVRRTTGDV |
| 3 | B22 | GRGSARQGVDY |
| 6 | Template | DKVTVWACQDN |

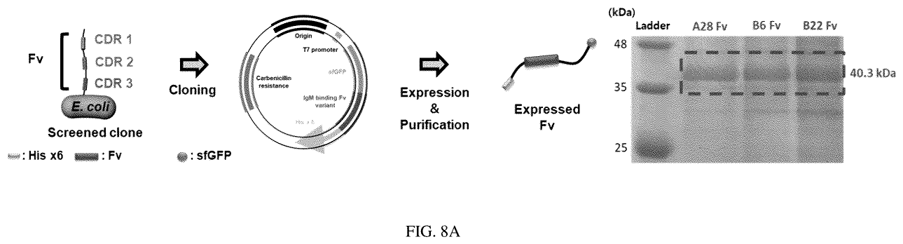

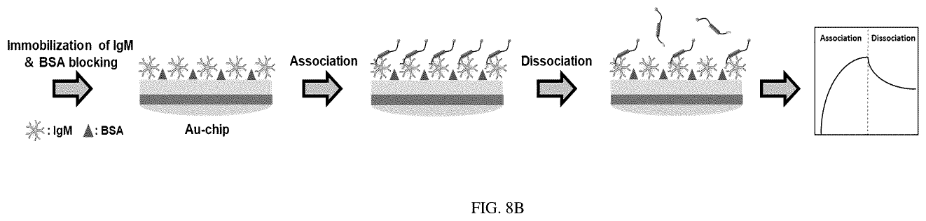

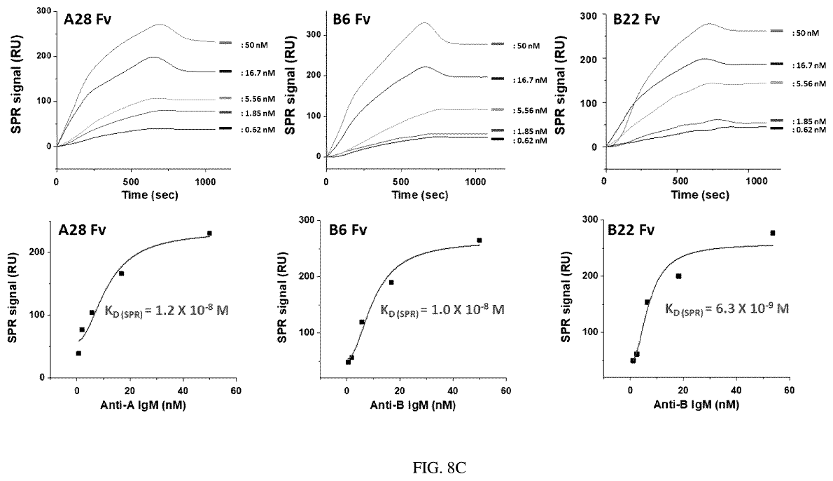

The selected Fv-antibody were expressed as fusion proteins with GFP. As shown in FIG. 8A, the Fv-antibody was composed of three CDRs including the screened CDR3 region and four FRs. The expression of the three Fv-antibody was observed to have a molecular weight of 40.3 kDa from SDS-PAGE. The association constants (KD) of the three expressed Fv antibodies on IgM were estimated using the SPR biosensor. As shown in FIG. 8B, IgM specific to A-type antigen (and B-type antigen) was immobilized on the gold surface of the SPR biosensor, and the expressed Fv-antibody were injected in the concentration range of 0.62 to 50 nM. As shown in FIG. 8C, the association-dissociation profiles were recorded according to the concentration of the expressed Fv-antibody. The association constant (KD) was calculated to be 12 nM (R2=0.960, n=3) for A28 Fv, 10 nM (R2=0.955, n=3) for B6 Fv, and 6.3 nM (R2=0.938, n=3) for B22 Fv. These results indicate 1) that Fv-antibody may strongly bind to IgM for not only B-type antigens but also A-type antigens with high affinity in the nanomolar range, and 2) suggest that Fv-antibody may be used as mimotopes of the A-type antigens and B-type antigens. A-type IgM: K_{D}=130 nM, B-type IgM: K_{D}=60 nM

### Evaluation of RBC Hemagglutination Inhibition Activity of Fv-Antibody

The purpose of selection and expression of Fv-antibody was to find mimotopes of A-type and B-type antigens in RBCs, and the Fv-antibody had binding affinity with IgM specific to these antigens. Mimotope activity was analyzed by estimating the RBC hemagglutination inhibition rate caused by mixing plasma with IgM having mismatching ABO types. RBC hemagglutination was estimated semiquantitatively using a gel card from BioRad Diagnostics (Hercules, CA, USA). The gel card was composed of a gel filtration resin through which the hemagglutinated RBCs could not penetrate.

The gel card experiment was performed through the following process.
A. 50 μL of ½⁷ diluted A-type anti-sera (Sihdia Shingyang Diagnostics, Siheung, Korea) and 50 μL of A28 Fv (0 to 24.8 μM), and 50 μL of ½⁷ diluted B-type anti-sera (Sihdia Shingyang Diagnostics, Siheung, Korea) and 50 μL of each of B6 Fv and B22 Fv (0 to 24.8 μM) reacted with each other in a rotator at 15 rpm for 1 hour at room temperature.
B. On purified IgM, 50 μL of A-type IgM 150 nM and 50 μL of A28 Fv (0 to 24.8 μM) and 50 μL of B-type IgM 150 nM and 50 μL of each of B6 Fv and B22 Fv (0 to 24.8 μM) reacted each other in a rotator at room temperature at 15 rpm for one hour.
C. In addition, to identify the selectivity of Fv, ½⁷ diluted anti-sera and purified IgM mismatching with Fv-antibody reacted therewith in a rotator at room temperature at 15 rpm for one hour.
D. 50 μL of ½⁷ diluted A-type anti-sera and 50 μL of A28 peptide (0 to 250 μM), 50 μL of ½⁷ diluted B-type anti-sera, and 50 μL of each of B6 peptide and B22 peptide (0 to 24.8 μM) reacted with each other in a rotator at room temperature at 15 rpm for one hour.
E. On purified IgM, 50 μL of 150 nM A-type IgM and 50 μL of A28 peptide (0 to 250 μM) and 50 μL of B-type IgM 150 nM and 50 μL of each of B6 Fv and B22 Fv (0 to 24.8 μM) reacted with each other in a rotator at room temperature at 15 rpm for one hour.
F. 1 mL of 0.8% Affirmagen A1 and B cells (Ortho-Clinical Diagnostics, Pencoed, UK) and 1 mL of ID-Diluent 2 (BioRad Diagnostics, Hercules, CA, USA) were mixed with each other, followed by stirring gently using a pipette.
G. 25 μL of prepared samples and 50 μL of prepared RBC were added to a gel card (ID-Card NaCl, Enzyme Test and Cold Agglutinins) (BioRad Diagnostics, Hercules, CA, USA), followed by centrifugation at 1030 rpm for 10 minutes using ID-Centrifuge 12 S II (BioRad Diagnostics, Hercules, CA, USA).

The hemagglutinated RBC was located at a top of the gel filtration resin (red). Non-hemagglutinated RBCs (red) that passed through the gel card tube could be observed under the gel card tube. Depending on the hemagglutination rate, RBCs (red color) that passed through the tube could be observed on a center of the tube, and the hemagglutination rate could be scored semi-quantitatively.

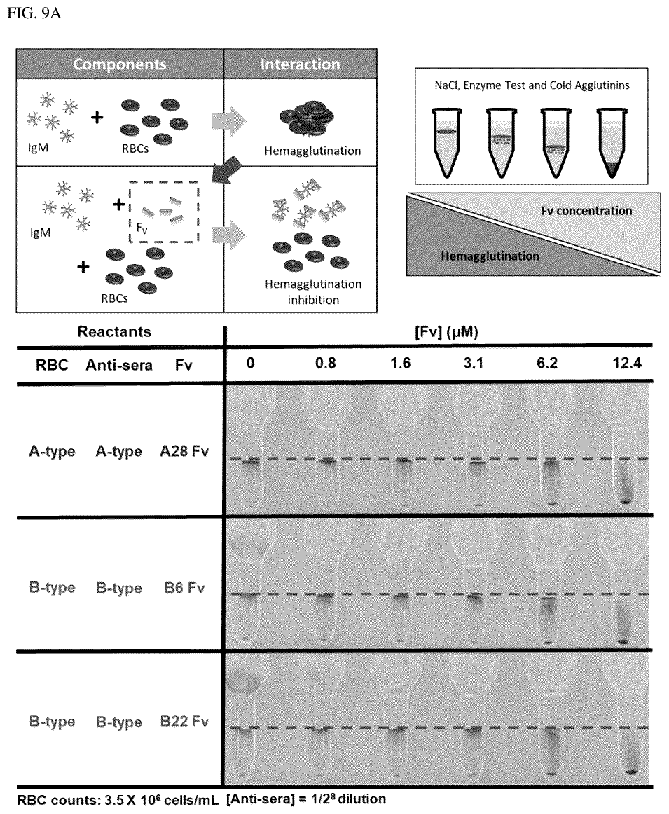

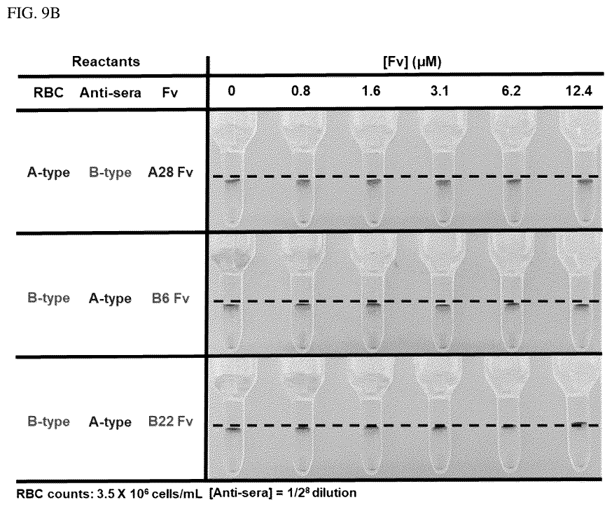

The inhibitory activity of the three expressed Fv-antibody was estimated by mixing different concentrations of expressed Fv-antibodies with the mixture of RBCs and the anti-sera mismatching therewith. As shown in FIG. 9A, a mixture of anti-sera (specific to A-type antigen and including IgM specific to A-type antigen) and RBCs (including A-type antigen) was prepared, and the mixture was treated with different concentrations of Fv-antibodies (A28 Fv). The hemagglutination of RBCs was observed at a low concentration (lane 1) of Fv-antibodies (A28 Fv) through the red RBCs at the top of the gel filtration resin. When the concentration of Fv-antibody increased, non-hemagglutinated RBCs were found between the top and bottom of the tube. For sufficiently high concentrations of Fv-antibodies (lane 6), RBC spots (red) were observed at the bottom of the tube. These results demonstrated that the hemagglutination of RBCs due to the anti-sera (including IgM specific to A-type antigen and specific to A-type antigen) mismatching therewith could be inhibited using the Fv-antibodies (A28 Fv). Therefore, the expressed Fv-antibody (A28Fv) could be used as the mimotope of the A-type antigen and could be used to inhibit the activity of IgM specific to the A-type antigen.

In addition, inhibition assays on mimotopes of the B-type antigen were performed by treating mixtures of anti-sera (specific to B-type antigen and including IgM specific to B-type antigen) and RBC (including B-type antigen) with different concentrations of Fv-antibodies (B6Fv and B22Fv). As shown in FIG. 9A, in the case of sufficiently high concentrations of Fv-antibodies (lane 6), hemagglutinated RBCs were found at a top of the gel filtration resin (lane 1). These results indicate that the expressed Fv-antibodies (B6 Fv and B22 Fv) may be used as mimotopes of the B-type antigen and may be used to inhibit IgM activity specific to B-type antigen.

The selectivity of mimotopes was estimated by measuring the IgM inhibition rate of mismatching ABO antigens. For example, when the mimotope of A-type antigen (A28 Fv) was treated with a mixture of anti-sera (specific to B-type antigen and including IgM specific to B-type antigen) and RBC (including B-type antigen), and then, hemagglutination of RBCs was observed. As shown in FIG. 9B, hemagglutination of RBCs was observed even at high concentrations of the mimotope of A-type antigen (A28 Fv) which was prevented from hemagglutination by the mimotope. The same selectivity was observed at the mimotope of B-type antigen (B6 Fv and B22 Fv). These results demonstrated that the mimotope of the A-type and B-type antigens may bind to IgM at the high affinity to prevent hemagglutination of RBCs and exhibit high selectivity to each IgM specific to each of the A-type and B-type antigen.

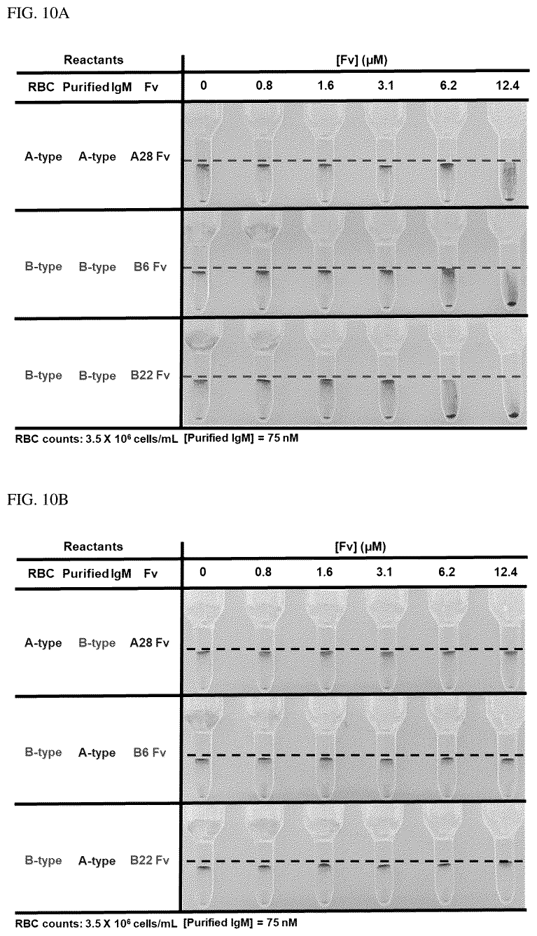

To identify the inhibitory activity from IgM in the antisera, the inhibition assay was performed using purified IgM instead of whole antisera. The inhibitory activity of the three synthesized Fvs was measured by mixing the Fv-antibody expressed at different concentrations with the mixture of RBC and mismatching IgM instead of anti-sera. As shown in FIG. 10A, a mixture of purified IgM (specific to A-type antigen) and RBC (including A-type antigen) was prepared, and the mixture was treated with different concentrations of Fv-antibodies (A28 Fv). At low concentrations of Fv-antibodies (A28 Fv) (lane 1), hemagglutination of RBCs was observed through the red RBCs at a top of the gel filtration resin. When the concentration of Fv-antibody was increased, non-hemagglutinated RBCs were found between the top and bottom of the tube. For sufficiently high concentrations of Fv-antibodies (lane 6), RBC spots (red) were observed at the bottom of the tube. These results demonstrated that the hemagglutination of RBCs (including A-type antigen) due to mismatching IgM (specific to A-type antigen) was prevented via inhibition of IgM (specific to A-type antigen) by mimotope (A28 Fv). In addition, inhibition assays on the mimotope of B-type antigen were performed by treating the mixture of IgM (specific to B-type antigen) and RBCs (including B-type antigen) with different concentrations of Fv-antibodies (B6Fv and B22Fv). As shown in FIG. 10A, for sufficiently high concentrations of Fv-antibodies (lane 6), hemagglutinated RBCs were found at the top of the gel filtration resin (lane 1). These results demonstrated that the expressed Fv-antibodies (B6Fv and B22Fv) could inhibit IgM (specific to B-type antigen) activity.

The selectivity of the mimotope was estimated by measuring the IgM inhibition rate of mismatching ABO antigens. For example, the mimotope of A-type antigen (A28 Fv) was treated with a mixture of IgM (against the B-type antigen) and RBC (using the B-type antigen), and the hemagglutination of RBCs was observed. As shown in FIG. 10B, the hemagglutination of RBCs was observed even with the mimotope of high concentration of A-type antigen (A28 Fv). The same selectivity was observed with the mimotope of B-type antigen (B6 Fv and B22 Fv). These results demonstrate that mimotopes of each of the A-type and B-type antigens may bind to IgM at the high affinity to prevent the RBC hemagglutination and exhibit high selectivity to each IgM specific to each of the A-type and B-type antigens.

### Evaluation of RBC Hemagglutination Inhibition Activity of Peptides

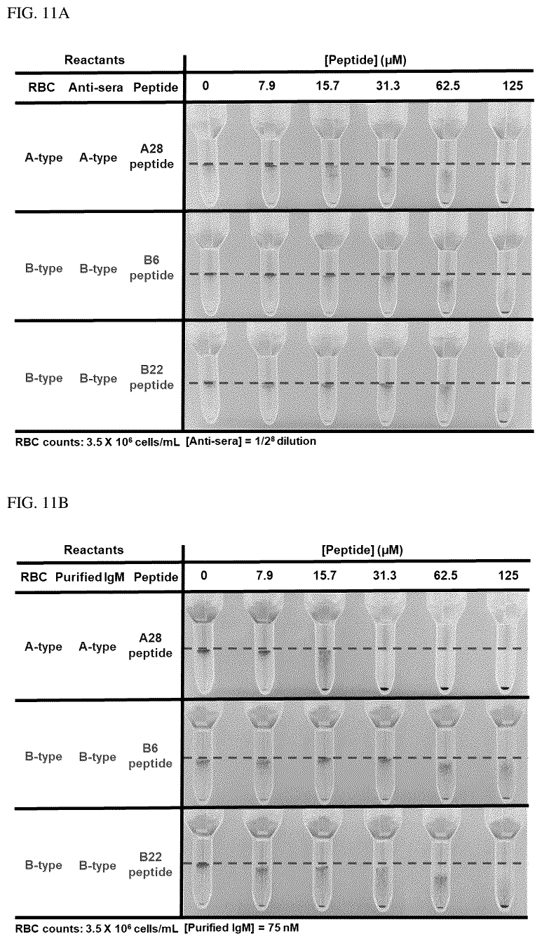

The selected Fv-antibody were identified to inhibit the activity of IgM specific to each of the A-type and B-type antigens for RBC hemagglutination. As mentioned above, the Fv library was prepared by site-directed mutagenesis of the CDR3 region, and the binding of the selected Fv-antibody occurred through the CDR3 region. To determine whether the binding of the CDR3 region may inhibit RBC hemagglutination, the CDR3 region in Table 2 was synthesized with a peptide (15 residues) and the inhibitory activity thereof was estimated. The inhibitory activity of the expressed three Fv-antibody was estimated by mixing different concentrations of synthetic CDR3 peptides with the mixture of RBC and mismatching anti-sera. As shown in FIG. 11A, a mixture of anti-sera (specific to A-type antigen and including IgM specific to A-type antigen) and RBC (including A-type antigen) was prepared, and the mixture was treated with the different concentrations of the synthesized CDR3 peptides (A28 peptide). At a low concentration of the synthesized CDR3 peptide (A28 peptide) (lane 1), hemagglutination of RBCs was observed through red RBCs at the top of the gel filtration resin. When the concentration of the synthesized CDR3 peptide (A28 peptide) was increased, non-hemagglutinated RBCs were found between the top and bottom of the tube. When the concentration of the synthesized CDR3 peptide (lane 6) was sufficiently high, RBC spots (red) were observed at the bottom of the tube. These results demonstrated that the synthesized CDR3 peptide (A28 peptide) could be used to inhibit the hemagglutination of RBCs due the mismatching anti-sera (specific to A-type antigen, and including IgM specific to A-type antigen). Therefore, the synthesized CDR3 peptide (A28 peptide) could be used as the mimotope of A-type antigen and could be used to inhibit the activity of IgM (specific to A-type antigen). In addition, the inhibition assay on the mimotope of B-type antigen was performed by treating the mixture of anti-sera (specific to B-type antigen and including IgM specific to B-type antigen) and RBC (including B-type antigen) with different concentrations of the synthesized CDR3 peptides (B6 peptide and B22 peptide). As shown in FIG. 11A, when the concentration of the synthesized CDR3 peptide (lane 6) was sufficiently high, hemagglutinated RBCs were found at the top of the gel filtration resin (lane 1). These results indicate that the expressed CDR3 peptides (B6 peptide and B22 peptide) may be used as mimotope of B-type antigen and may be used to inhibit IgM (specific to B-type antigen) activity.

To identify whether the inhibitory activity was generated from purified IgM in the antiserum, the inhibition assay was performed using purified IgM rather than whole antiserum. The inhibitory activities of the three synthesized CDR3 peptides were estimated by mixing different concentrations of the synthesized CDR3 peptides with the mixture of RBC and mismatching IgM instead of anti-sera. As shown in FIG. 11B, the mixture of purified IgM (against A-type antigen) and RBC (with A-type antigen) was prepared and treated with different concentrations of the synthesized CDR3 peptides (A28 peptide). At low concentrations of the synthesized CDR3 peptide (A28 peptide) (lane 1), hemagglutination of RBCs was observed through red RBCs at the top of the gel filtration resin. When the concentration of Fv-antibody was increased, non-hemagglutinated RBCs were found between the top and bottom of the tube. When the concentration of the synthesized CDR3 peptide (lane 6) was sufficiently high, RBC spots (red) were observed at the bottom of the tube. These results demonstrated that the hemagglutination of RBCs (including A-type antigen) due to mismatching IgM (specific to A-type antigen) was prevented via the inhibition of IgM (specific to A-type antigen) by mimotope (A28 peptide). In addition, the inhibition assay on the mimotope of the B-type antigen was performed by treating the mixture of IgM (specific to B-type antigen) and RBC (including B-type antigen) with different concentrations of the synthesized CDR3 peptides (B6 peptide and B22 peptide). As shown in FIG. 11B, in the case of sufficiently high concentration of Fv-antibody (lane 6), hemagglutinated RBCs were found at the top of the gel filtration resin (lane 1). These results demonstrated that the synthesized CDR3 peptides (B6 peptide and B22 peptide) could inhibit the activity of IgM (specific to B-type antigen).

### Example 1

Fv-1 was coated on a MaxiSorp plate with high protein binding ability using a solution containing Fv-1 at a concentration of 50 μg/ml, followed by washing using a PBS solution. Subsequently, a solution including BSA at a concentration of 5 mg/ml was applied thereto, followed by washing using a PBS solution. Subsequently, a 100 nM switching peptide solution was applied thereto, and then kept at room temperature for 1 hour to bind the switching peptide to Fv-1, followed by washing using a PBS solution. Subsequently, 100 μL of a sample solution including immunoglobulin at each of concentrations of 0, 1 pg/ml, 10 pg/ml, 100 pg/ml, 1 ng/ml, 10 ng/ml, 100 ng/ml, and 1 μg/ml was applied thereto, and followed by reaction at room temperature for 1 hour, and then fluorescence was measured.

### Example 2

An experiment was performed in the same manner as in Example 1, except that Fv-2 was coated on a MaxiSorp plate using a solution containing Fv-2 at a concentration of 50 μg/ml, and then fluorescence was measured.

### Example 3

An experiment was performed in the same manner as in Example 1, except that Fv-2 was coated on a MaxiSorp plate using a solution containing Fv-3 at a concentration of 50 μg/ml, and then fluorescence was measured.

### Experimental Example

### IgM Collection Experiment

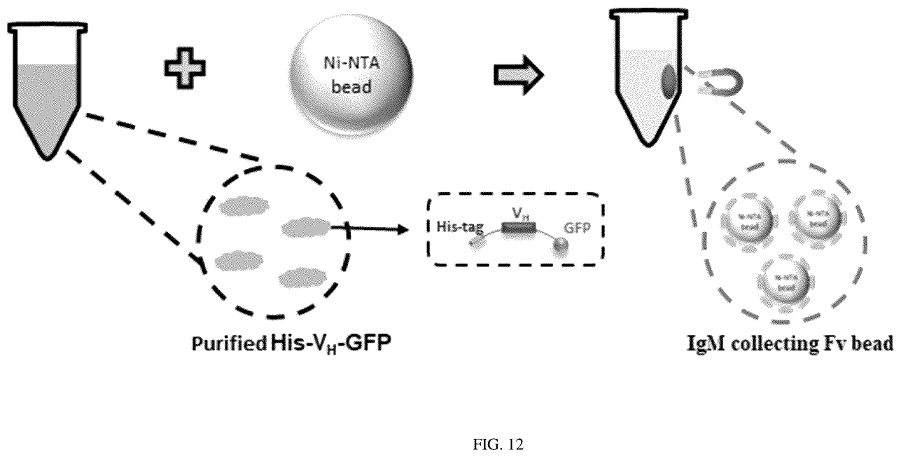

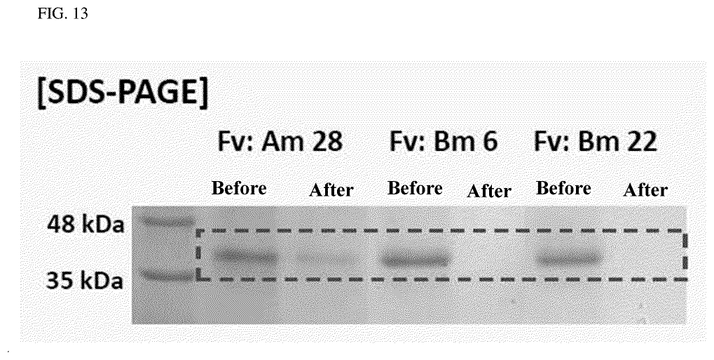

Purified His-VH-GFP in which His-tag, VH, and GFP were linked to each other was prepared to prepare a supernatant. Ni-NTA beads including nickel were prepared and mixed with the His-VH-GFP. His-tag binds to the beads, thereby forming Fv beads capable of collecting IgM. The formation process is illustrated in FIG. 12. The above reaction was verified using SDS-page, and the results are illustrated in FIG. 13. As Fv binds to the beads, the amount of Fv in the supernatant decreases. Based on a result of measuring the concentration of the supernatant before reacting with the beads and the concentration of the supernatant after reacting with the beads using the BCA kit, it is identified that 3 μg of Fv binds to per 1 μL of beads.

After mixing the beads with a solution including immunoglobulin, the beads were removed therefrom using a magnetic field. It was expected that in state in which immunoglobulin binds to the beads, the beads are separated therefrom, and the process is shown in FIG. 14. The Anti-A IgM purified through this process, diluted Antisera A, and Anti-A IgM in B-type human plasma were collected using Am 28 Fv beads, and the purified Anti-B IgM, diluted Antisera B, and Anti-B IgM in A-type human plasma were collected using Bm 6 and Bm 22 Fv beads.

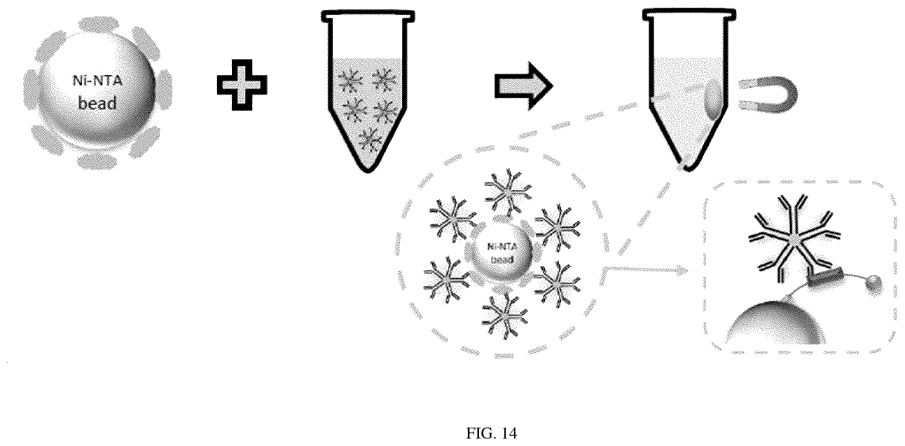

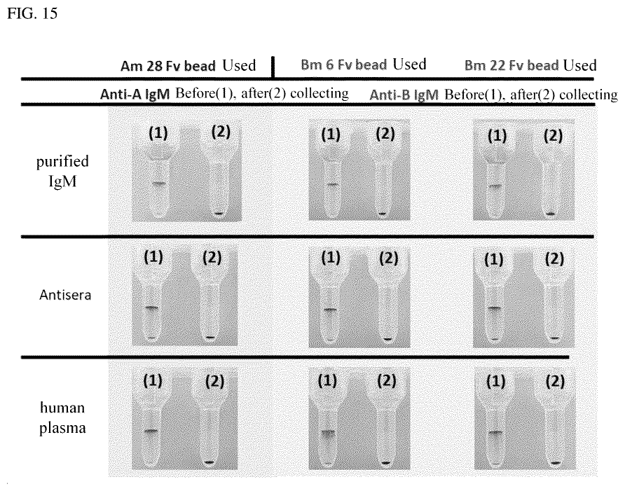

After the collection, it was identified that the hemagglutination of red blood cells was inhibited using a gel card. The experimental conditions were as follows: Fv beads 20 μL, [purified IgM]=75 nM, Antisera 1/256), human plasma=100 μL, and the reaction was performed at room temperature for 1 hour. The results are shown in FIG. 15. It may be identified that the target of the Am 28 Fv bead is Anti-A IgM, the target of the Bm 6 Fv bead is Anti-B IgM, and the target of the Bm 22 Fv bead is Anti-B IgM.

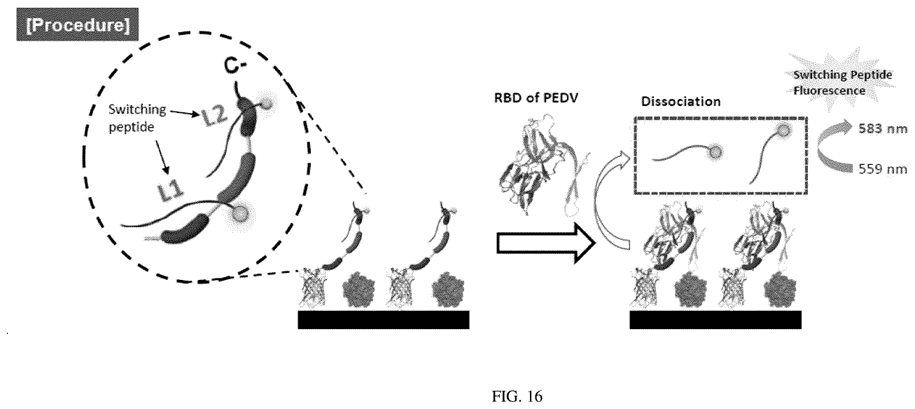

One-step immunoassay is performed. The experimental procedure is as follows: Fv coating on a maxisorp plate (50 ug/ml, 4 C, overnight), PBS washing 3×, BSA blocking (5 mg/ml, RT, 30 min), PBS washing 3×, Switching Peptide binding: L1-peptide 100 nM (RT, 1 h), PBS washing 1×, RBD (or PED virus) binding (RT, 1 h), RBD: 1 pg/ml-100 ng/ml ( 1/10 dilution), PEDV: 1 ug/mL (100 μL), Measure dissociated L2-FAM fluorescence (Ex/Em=488/521 nm) by victor×5. The above procedure is schematically illustrated in FIG. 16.

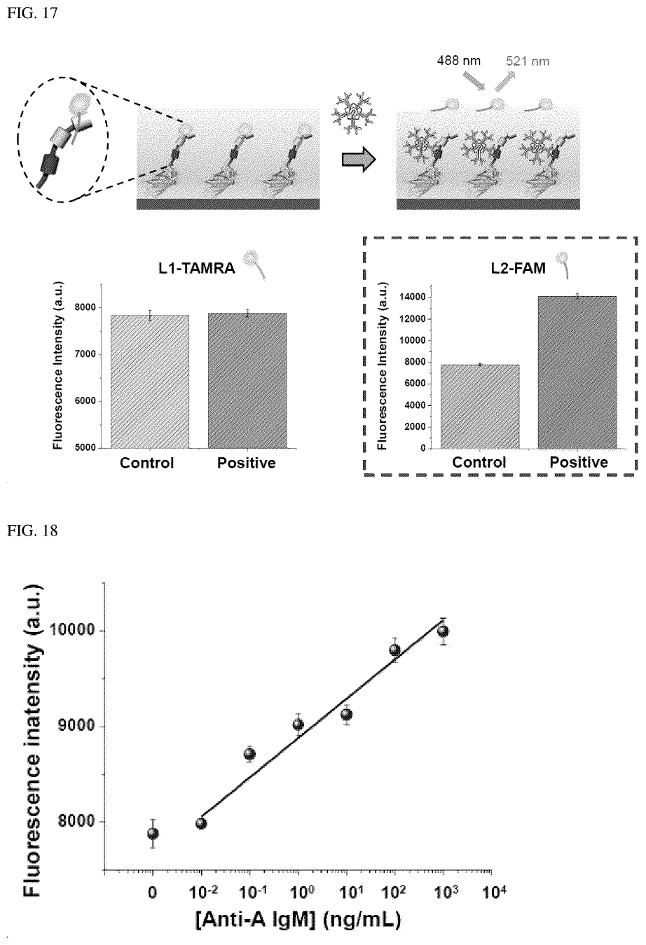

The switching peptide for A28 Fv activity test was selected. The schematic experimental procedure and results are illustrated in FIG. 17. Referring to FIG. 17, it may be identified that there was no difference in fluorescence intensity between the control group and the L1-TAMRA switching peptide, while the L2-FAM switching peptide exhibited a high fluorescence intensity, compared to the control group. Thus, the L2-FAM switching peptide was selected. Next, the fluorescence intensity based on the concentration of Anti-A IgM was identified. The general experimental procedure is as follows: Fv coating on a maxisorp plate (50 ug/ml, 4° C., overnight), PBS washing 3×, BSA blocking (5 mg/ml, RT, 30 min), PBS washing 3×, Switching Peptide binding: 300 nM (RT, 1 h), PBS washing 3×, Anri-A IgM binding (RT, 1 h) 100 pg/ml-10 ug/ml ( 1/10 dilution, 6 concentrations), Measure dissociated L1-TAMRA fluorescence (Ex/Em=552/578 nm) by victor×5. The experimental results are shown in FIG. 18. Referring to FIG. 18, it may be identified that the fluorescence intensity increases linearly with respect to the logarithmic scale of the anti-A IgM concentration.

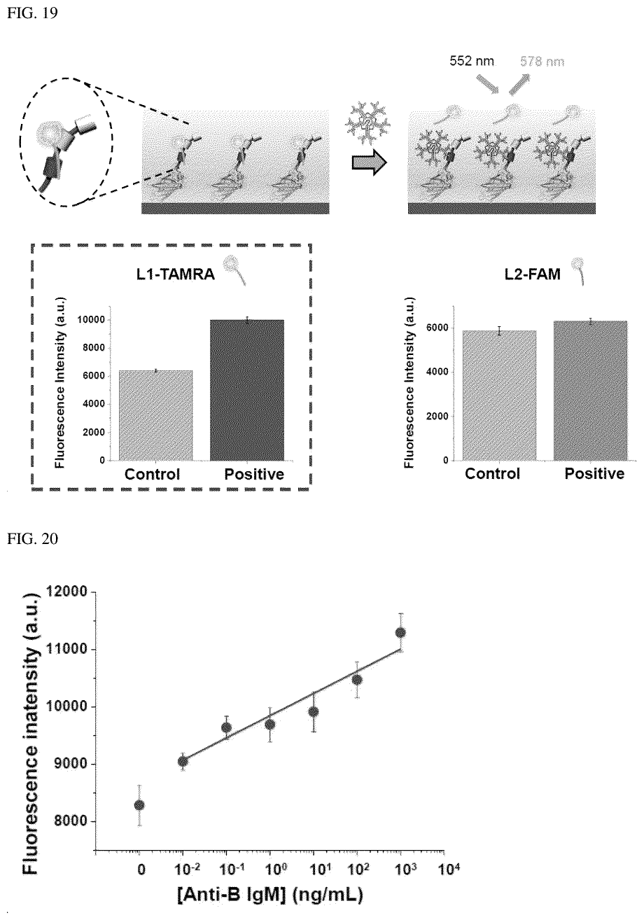

The switching peptide for the B6 Fv activity test was selected. The schematic experimental procedure and results are shown in FIG. 19. Referring to FIG. 19, it may be identified that there was no difference in fluorescence intensity between the control group and the L2-FAM switching peptide, while the L1-TAMRA switching peptide exhibited a high fluorescence intensity compared to the control group. Therefore, the L1-TAMRA switching peptide was selected. Next, the fluorescence intensity based on the concentration of Anti-B IgM was identified. The general experimental procedure is as follows: Fv coating on a maxisorp plate (50 ug/ml, 4° C., overnight), PBS washing 3×, BSA blocking (5 mg/ml, RT, 30 min), PBS washing 3×, Switching Peptide binding: 300 nM (RT, 1 h), PBS washing 3×, Anri-A IgM binding (RT, 1 h) 100 pg/ml-10 ug/ml ( 1/10 dilution, 6 concentrations), Measure dissociated L1-TAMRA fluorescence (Ex/Em=552/578 nm) by victor×5. The experimental results are shown in FIG. 20. Referring to FIG. 20, it may be identified that the fluorescence intensity increases linearly with respect to the logarithmic scale of the anti-B IgM concentration.

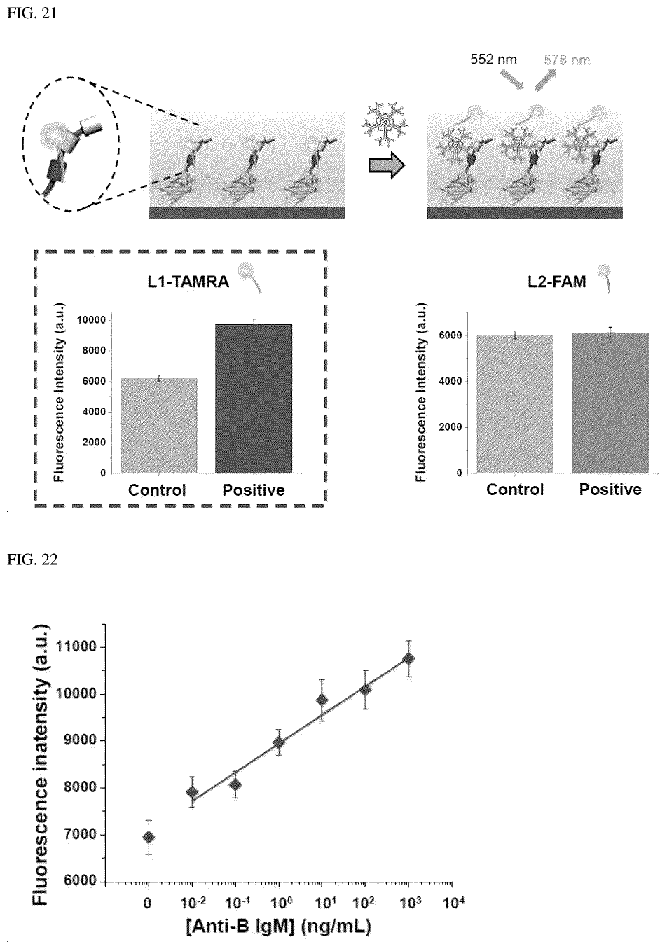

The switching peptide for the B22 Fv activity test was selected. The general experimental procedure and results are shown in FIG. 21. Referring to FIG. 21, it may be identified that there was no difference in fluorescence intensity between the control group and the L2-FAM switching peptide, but the L1-TAMRA switching peptide exhibited a high fluorescence intensity compared to the control group, and thus the L1-TAMRA switching peptide was selected. Next, the fluorescence intensity based on the concentration of Anti-B IgM was identified. The general experimental procedure is as follows: Fv coating on a maxisorp plate (50 ug/ml, 4° C., overnight), PBS washing 3×, BSA blocking (5 mg/ml, RT, 30 min), PBS washing 3×, Switching Peptide binding: 300 nM (RT, 1 h), PBS washing 3×, Anri-A IgM binding (RT, 1 h) 100 pg/ml-10 ug/ml ( 1/10 dilution, 6 concentrations), Measure dissociated L1-TAMRA fluorescence (Ex/Em=552/578 nm) by victor×5. The experimental results are shown in FIG. 22. Referring to FIG. 22, it may be identified that the fluorescence intensity increases linearly with respect to the logarithmic scale of the anti-B IgM concentration.

Although embodiments of the present disclosure have been described with reference to the accompanying drawings, the present disclosure is not limited to the above embodiments, but may be implemented in various different forms. A person skilled in the art may appreciate that the present disclosure may be practiced in other concrete forms without changing the technical spirit or essential characteristics of the present disclosure. Therefore, it should be appreciated that the embodiments as described above is not restrictive but illustrative in all respects.

What is claimed is:

1. An immunoassay device for detecting blood type antigen-specific immunoglobulin, the immunoassay device comprising:
a substrate having a reaction space defined therein capable of receiving therein a detection target sample solution;
a binding antibody positioned in the reaction space and immobilized on the substrate;
a switching peptide having a peptide compound reversibly binding to a Fab region of the binding antibody and a fluorescent label binding to the peptide compound; and
a fluorescence-quenching substance positioned adjacent to the fluorescent label and quenching fluorescence from the fluorescent label,
wherein the binding antibody includes one selected from the group consisting of a CDR3 including an amino acid sequence of SEQ ID NO 1, a CDR3 including an amino acid sequence of SEQ ID NO 2, and a CDR3 including an amino acid sequence of SEQ ID NO 3, and thus specifically binds to the blood type antigen-specific immunoglobulin.

2. The immunoassay device of claim 1, wherein the binding antibody includes one selected from the group consisting of:
a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 1;
a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 2; and
a heavy chain variable region including a CDR1 including an amino acid sequence of SEQ ID NO 4, a CDR2 including an amino acid sequence of SEQ ID NO 5, and a CDR3 including an amino acid sequence of SEQ ID NO 3.

3. The immunoassay device of claim 2, wherein the peptide compound has an amino acid sequence capable of specifically and reversibly binding to at least one of first to fourth framework regions of a light chain or a heavy chain of the binding antibody.

4. The immunoassay device of claim 2, wherein the binding antibody includes one selected from the group consisting of:
a heavy chain variable region including an amino acid sequence of SEQ ID NO 1;
a heavy chain variable region including an amino acid sequence of SEQ ID NO 2; and
a heavy chain variable region including an amino acid sequence of SEQ ID NO 3.

5. The immunoassay device of claim 1, wherein the immunoassay device further comprises:
a light source for irradiating light to the detection sample solution received in the reaction space of the substrate; and
an image analysis device configured to receive fluorescence generated from the switching peptide released from the binding antibody, to generate an image of the received fluorescence, and to analyze the image to analyze an amount of the switching peptide released from the binding antibody.

6. The immunoassay device of claim 1, wherein the blood type antigen-specific immunoglobulin is A-type antigen-specific IgM or B-type antigen-specific IgM.

7. The immunoassay device of claim 1, wherein the fluorescent label includes at least one selected from the group consisting of rhodamine and derivatives thereof, fluorescein and derivatives thereof, coumarin and derivatives thereof, acridine and derivatives thereof, pyrene and derivatives thereof, erythrosine and derivatives thereof, eosin and derivatives thereof, 4-acetamido-4′-isothiocyanatostilbene-2,2′ disulfonic acid, fluorescein isothiocyanate (FITC), oregon green, Alex Fluoro, carboxyfluorescein (FAM), 6-carboxy-4′,5′-dichloro-2′,7′-dimethoxyfluorescein (JOE), carboxy-X-rhodamine (ROX), 6-carboxy-2′,4,4′,5′,7,7′-hexachlorofluorescein (HEX), Texas Red (sulforhodamine 101 acid chloride), 6-carboxy-2′,4,7′,7-tetrachlorofluorescein (TET), tetramethylrhodamine-isothiocyanate (TRITC), carboxytetramethylrhodamine (TAMRA), cyanine-based dyes, and siadicarbocyanine dyes.

8. The immunoassay device of claim 1, wherein the fluorescence-quenching substance includes at least one selected from the group consisting of 4-(dimethylamino) azobenzene-4-carboxylic acid (DABCYL), 4-(dimethylamino) azobenzene sulfonic acid (DABSYL), blackhole quencher (BHQ), blackberry quencher (BBQ), ECLIPSE quencher, Tide quencher, a carbon nanomaterial, and a manganese dioxide nanomaterial.

9. An immunoassay method for detecting blood type antigen-specific immunoglobulin, the method comprising:
a first step of immobilizing a binding antibody having a switching peptide binding thereto to a substrate or immobilizing the binding antibody to the substrate, and then binding the switching peptide to the binding antibody, and binding a quenching substance to the binding antibody or the substrate;
a second step of treating the binding antibody with a detection target sample solution; and
a third step of irradiating light to the detection target sample solution after the treatment, and quantitatively analyzing a target antigen in the detection target sample solution based on fluorescence generated from a fluorescent label of the switching peptide released from the binding antibody,
wherein the binding antibody includes one selected from the group consisting of a CDR3 including an amino acid sequence of SEQ ID NO 1, a CDR3 including an amino acid sequence of SEQ ID NO 2, and a CDR3 including an amino acid sequence of SEQ ID NO 3, and thus specifically binds to the blood type antigen-specific immunoglobulin,
wherein the switching peptide includes a peptide compound having an amino acid sequence of capable selectively and reversibly binding to a Fab (Fragment antigen-binding) region of the binding antibody, and the fluorescent label binding thereto and emitting fluorescent light.

10. The immunoassay method of claim 9, wherein in the second step, when the blood type antigen-specific immunoglobulin is present in the detection target sample solution, and thus the blood type antigen-specific immunoglobulin binds to the binding antibody, the switching peptide is quantitatively released from the binding antibody depending on an amount of the blood type antigen-specific immunoglobulin having reacted with the binding antibody.

11. A method for collecting blood type antigen-specific immunoglobulin, the method comprising:
adding a bead having an antibody or a peptide immobilized on a surface thereof specifically binding to the blood type antigen-specific immunoglobulin into a sample containing the blood type antigen-specific immunoglobulin to induce a reaction between the blood type antigen-specific immunoglobulin and the antibody or the peptide; and
collecting the bead from the sample,
wherein the binding antibody includes one selected from the group consisting of a CDR3 including an amino acid sequence of SEQ ID NO 1, a CDR3 including an amino acid sequence of SEQ ID NO 2, and a CDR3 including an amino acid sequence of SEQ ID NO 3, and thus specifically binds to the blood type antigen-specific immunoglobulin.

12. The collecting method of claim 11, wherein the bead includes nickel (Ni), copper (Cu), zinc (Zn), or cobalt (Co)-containing magnetic particles having a chelating group including nitrilotriacetic acid (NTA) or iminodiacetic acid (IDA) modified on surfaces thereof.

13. The collecting method of claim 12, wherein the bead is made of a magnetic material,
wherein the bead is collected from the sample using a magnetic field.
